(19) United States
(12) Patent Application Publication
(10) Pub. No.: US 2007/0270362 A1
(43) Pub. Date: Nov. 22, 2007

(54) Methods and compositions for prevention or treatment of inflammatory-related diseases and disorders

(75) Inventors: John M. Harlan, Seattle, WA (US); Lynn M. Schnapp, Seattle, WA (US); Barbara R. Schwartz, Seattle, WA (US); Li Liu, Sammamish, WA (US); Elaine White Raines, Seattle, WA (US); Yoshiaki Tsubota, Seattle, WA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: The University of Washington, Seattle, WA (US)

(21) Appl. No.: 11/437,977

(22) Filed: May 18, 2006

(51) Int. Cl. A61K 48/00; A61K 31/55; A61K 39/395; A61K 31/522; A61K 31/517

(52) U.S. Cl. 514/44; 514/263.32; 514/414; 514/216; 514/214.01; 514/266.1; 424/133.1; 514/2

(57) ABSTRACT

Methods and compositions are provided for treating inflammatory-related diseases and related disorders in a mammalian subject. The method provides administering to the mammalian subject one or more modulators of cyclin dependent kinase 4 (Cdk4) activity in an amount effective to reduce or eliminate the inflammatory-related disease or disorder or prevent its occurrence or recurrence. Methods are also provided for reducing or eliminating ligand-induced adhesion in a mammalian subject. The method provides administering to the subject one or more modulators of cyclin dependent kinase 4 (Cdk4) activity in an amount effective to reduce or eliminate ligand-induced adhesion.

### STATEMENT OF GOVERNMENT SUPPORT

This invention was made by government support by Grant Nos. NIH HL18645 and HL67267 from the National Institutes of Health. The Government has certain rights in this invention.

### FIELD

The invention relates to the field of pharmaceutical agents and, more specifically, is directed to modulators, compositions, uses and methods for treating inflammatory-related diseases and disorders.

### BACKGROUND

Protein kinases represent a large family of enzymes, which catalyze the phosphorylation of target protein substrates. The phosphorylation is usually a transfer reaction of a phosphate group from ATP to the protein substrate. Common points of attachment for the phosphate group to the protein substrate include, for example, a tyrosine, serine or threonine residue. For example, protein tyrosine kinases (PTKs) are enzymes, which catalyze the phosphorylation of specific tyrosine residues in cellular proteins. Examples of kinases in the protein kinase family include, without limitation, ab1, Akt, Aurora-A, Aurora-B, bcr-ab1, Blk, Brk, Btk, c-kit, c-Met, c-src, c-fms, CDK1, CDK2, CDK3, CDK4, CDK5, CDK6, CDK7, CDK8, CDK9, CDK10, cRaf1, CSF1R, CSK, EGFR, ErbB2, ErbB3, ErbB4, Erk, Fak, fes, FGFR1, FGFR2, FGFR3, FGFR4, FGFR5, Fgr, flt-1, Fps, Frk, Fyn, Hck, IGF-1R, INS-R, Jak, KDR, Lck, Lyn, MEK, p38, PDGFR, PIK, PKC, PYK2, ros, tie, tie2, TRK, Yes, and Zap70. Due to their activity in numerous cellular processes, protein kinases have emerged as important therapeutic targets.

Leukocyte adhesion and migration plays a key role in response to inflammation and infection. This process is a well-orchestrated series of adhesion, de-adhesion, signaling and cytoskeletal changes that is tightly regulated. Leukocytes do not adhere to underlying endothelial cells when in a resting state. However, upon activation, i.e., by cytokines or chemokines, leukocytes rapidly modulate changes in integrin conformation and/or clustering to alter integrin affinity and/or avidity that permit targeted integrin-mediated adhesion to the vascular endothelial cells and subsequent transmigration. However, an inappropriate or overexuberant leukocyte response may cause significant tissue damage.

The elucidation of the intricacy of protein kinase pathways and the complexity of the relationship and interaction among and between the various protein kinases and kinase pathways highlights the importance of developing pharmaceutical agents capable of acting as protein modulators, regulators or inhibitors that have beneficial activity thereby modulating various disease states.

A need exists to develop new therapeutic treatments that are useful in treating various conditions associated with protein kinase activation conditions associated with inappropriate leukocyte accumulation. For many of these conditions the currently available treatment options are inadequate.

### SUMMARY

A method for preventing or treating inflammatory-related diseases and related disorders in a mammalian subject is provided comprising administering to the subject one or more modulators of cyclin dependent kinase 4 (Cdk4) activity in an amount effective to reduce or eliminate the inflammatory-related disease or disorder or prevent its occurrence or recurrence. The target of the modulator is cyclin dependent kinase 4 (Cdk4). The modulator can be an inhibitor and an inhibitor can be a small chemical compound, peptide or peptide deriviatives, siRNA, ribozyme, antisense, or antibody.

In one aspect, the invention provides a method for treating an inflammatory-related disease or disorder in a mammalian subject comprising administering to the subject one or more modulators of cyclin dependent kinase 4 (Cdk4) activity in an amount effective to reduce or eliminate the inflammatory-related disease or disorder or prevent its occurrence or recurrence. In some methods, the Cdk4 activity regulates ligand-induced adhesion (LIA). In other methods, the LIA is integrin-mediated. In some such methods, the LIA is small GTPase Rap-1 independent. In some methods, the Cdk4 modulator inhibits integrin-mediated adhesion in leukocytes. In other methods the Cdk4 modulator inhibits integrin-mediated adhesion in monocytes. In some methods, the modulator is a small chemical compound, short interfering RNA, dominant-negative molecule, short hairpin RNA, ribozyme, antisense oligonucleotide, antibody, peptide or peptidomimetic. In some such methods, the dominant-negative molecule is a dominant-negative peptide or peptidomimetic. In other such methods, the small chemical compound is flavopiridol, indolinone, oxindole 91, N6-isopentenyladenine, olomoucine, (R)-Roscovitine, hymenialdisine, fascaplysin, compound 66, purvalanol A, purvalanol B, indirubin-3′monoxime, indirubin-5-sulfonate, SU9516, compound 26a, compound 15b, alsterpaullone, quinazoline 51, CINK4, PD0183812, NSC 625987, PD 0332991, aminopurvalanol, and Calyculin phosphatase inhibitor. In other methods, the subject is human. In some methods, the inflammatory-related disease or disorder is the inflammatory-related disease or disorder include, but not limited to diabetes (such as Type II diabetes, Type I diabetes, diabetes insipidus, diabetes mellitus, maturity-onset diabetes, juvenile diabetes, insulin-dependant diabetes, non-insulin dependant diabetes, malnutrition-related diabetes, ketosis-prone diabetes or ketosis-resistant diabetes); nephropathy (such as glomerulonephritis or acute/chronic kidney failure); obesity (such as hereditary obesity, dietary obesity, hormone related obesity or obesity related to the administration of medication); hearing loss (such as that from otitis extema or acute otitis media); fibrosis related diseases (such as pulmonary interstitial fibrosis, renal fibrosis, cystic fibrosis, liver fibrosis, wound-healing or burn-healing, wherein the burn is a first-, second- or third-degree burn and/or a thermal, chemical or electrical burn); arthritis (such as rheumatoid arthritis, rheumatoid spondylitis, osteoarthritis or gout); an allergy; allergic rhinitis; acute respiratory distress syndrome; asthma; bronchitis; an inflammatory bowel disease (such as irritable bowel syndrome, mucous colitis, ulcerative colitis, Crohn's disease, gastritis, esophagitis, pancreatitis or peritonitis); or an autoimmune disease (such as scleroderma, systemic lupus erythematosus, myasthenia gravis, transplant rejection, endotoxin shock, sepsis, psoriasis, eczema, dermatitis or multiple sclerosis).

In another aspect, the invention provides a method for reducing or eliminating ligand-induced adhesion in a mammalian subject comprising administering to the subject one or more modulators of cyclin dependent kinase 4 (Cdk4) activity in an amount effective to reduce or eliminate ligand-induced adhesion. In some methods, the modulator is a small chemical compound, short interfering RNA, dominant-negative molecule, short hairpin RNA, ribozyme, antisense oligonucleotide, antibody, peptide or peptidomimetic. In other such methods, the dominant-negative molecule is a dominant-negative peptide or peptidomimetic. In some such methods, the small chemical compound is flavopiridol, indolinone, oxindole 91, N6-isopentenyladenine, olomoucine, (R)-Roscovitine, hymenialdisine, fascaplysin, compound 66, purvalanol A, purvalanol B, indirubin-3′monoxime, indirubin-5-sulfonate, SU9516, compound 26a, compound 15b, alsterpaullone, quinazoline 51, CINK4, PD0183812, NSC 625987, PD 0332991, aminopurvalanol, and Calyculin phosphatase inhibitor. In other methods the subject is human.

In another aspect, the invention provides a composition comprising a therapeutically effective amount of at least one modulator of cyclin dependent kinase 4 (Cdk4) activity for treatment of an inflammatory-related disease or disorder in a mammalian subject. In some compositions, therapeutically effective amount is a prophylactically effective amount. In other compositions, the Cdk4 activity regulates ligand-induced adhesion (LIA). In some such compositions, the LIA is integrin-mediated. In other such compositions, the LIA is small GTPase Rap-1 independent. In some compositions, wherein the Cdk4 modulator inhibits integrin-mediated adhesion in leukocytes. In other compositions, the Cdk4 modulator inhibits integrin-mediated adhesion in monocytes.

In another aspect, the invention provides a pharmaceutical composition comprising at least one Cdk4 modulator for treatment of an inflammatory-related disease or disorder in a mammalian subject and a pharmaceutically acceptable carrier. In some pharmaceutical compositions, the inflammatory-related disease or disorder include, but not limited to diabetes (such as Type II diabetes, Type I diabetes, diabetes insipidus, diabetes mellitus, maturity-onset diabetes, juvenile diabetes, insulin-dependant diabetes, non-insulin dependant diabetes, malnutrition-related diabetes, ketosis-prone diabetes or ketosis-resistant diabetes); nephropathy (such as glomerulonephritis or acute/chronic kidney failure); obesity (such as hereditary obesity, dietary obesity, hormone related obesity or obesity related to the administration of medication); hearing loss (such as that from otitis extema or acute otitis media); fibrosis related diseases (such as pulmonary interstitial fibrosis, renal fibrosis, cystic fibrosis, liver fibrosis, wound-healing or burn-healing, wherein the burn is a first-, second- or third-degree burn and/or a thermal, chemical or electrical burn); arthritis (such as rheumatoid arthritis, rheumatoid spondylitis, osteoarthritis or gout); an allergy; allergic rhinitis; acute respiratory distress syndrome; asthma; bronchitis; an inflammatory bowel disease (such as irritable bowel syndrome, mucous colitis, ulcerative colitis, Crohn's disease, gastritis, esophagitis, pancreatitis or peritonitis); or an autoimmune disease (such as scleroderma, systemic lupus erythematosus, myasthenia gravis, transplant rejection, endotoxin shock, sepsis, psoriasis, eczema, dermatitis or multiple sclerosis). In some pharmaceutical compositions, the modulator is interfering RNA, short hairpin RNA, ribozyme, antisense oligonucleotide, or protein inhibitor. In other pharmaceutical compositions, the modulator is a dominant-negative molecule, peptide, peptidomimetic or a small chemical molecule. In some such pharmaceutical compositions, the dominant-negative molecule is a dominant-negative peptide or peptidomimetic. In other such pharmaceutical compositions, the small chemical compound is flavopiridol, indolinone, oxindole 91, N6-isopentenyladenine, olomoucine, (R)-Roscovitine, hymenialdisine, fascaplysin, compound 66, purvalanol A,B, indirubin-3′monoxime, indirubin-5-sulfonate, SU9516, compound 26a, compound 15b, alsterpaullone, quinazoline 51, CINK4, PD0183812, NSC 625987, PD 0332991, aminopurvalanol, and Calyculin phosphatase inhibitor. The pharamaceutical compositions can be administered orally, topically or systemically.

### BRIEF DESCRIPTION OF THE DRAWINGS

The file of this patent contains at least one drawing/photograph executed in color. Copies of this patent with color drawing(s)/photograph(s) will be provided by the Office upon request and payment of the necessary fee.

FIG. 1. Jurkat cell adhesion to endothelial cells and EC matrix. A. Adhesion of unstimulated Jurkat cells (green) to confluent monolayer of BAEC (red) after 30 min. B. Adhesion of unstimulated Jurkat cells to edge of wounded BAEC monolayer. Confluent endothelial monolayer was wounded by scratching and intervening matrix was removed by scraping and aspirating. C. Adhesion of unstimulated Jurkat cells to cytochalasin-retracted BAEC monolayer. D. Adhesion of unstimulated Jurkat cells to BAEC-derived matrix. EC matrix was prepared by treating confluent EC monolayer with 20 mM NH₄OH at 37° C. for 5 min. Fibronectin fibrils are visualized in red.

FIG. 2. Unstimulated lymphocytes adhere to retracted endothelial cells and to endothelial cell matrix. A. Jurkat cells, Ramos cells or PBMC were allowed to adhere to BAEC, HUVEC or EC-derived matrix for 20 min at 37° C. EC treatments were: human thrombin, 10 U/ml×10 min; cytochalasin D 1 μM×75 min; latrunculin A, 1 μM×50 min. Ordinate: fold-increase in adhesion compared to adhesion to untreated EC monolayer of at least 6 independent experiments done in triplicate. p<0.01 for all compared to controls. B. Jurkat cell adhesion to thrombin or cytochalasin-D retracted BAEC or untreated BAEC monolayer (control). Representative experiment is shown. Data are average of 6 replicate wells ±SD.

FIG. 3. Ligand-induced adhesion is dependent on β1 integrins. A. Jurkat cells were pretreated with indicated function blocking antibody for 10 minutes, then allowed to adhere to cytochalasin D-retracted BAEC for 20 min. B. Jurkat cells were pretreated with indicated function blocking antibody for 10 minutes, then allowed to adhere to BAEC-derived matrix for 20 min. Adhesion index is reported as the ratio of adhesion with antibodies/adhesion without antibodies of 6 replicate wells ±SD.

FIG. 4. A. Cdk inhibitors block ligand-induced adhesion. Jurkat cells or PBMC were treated with 20 μM roscovitine, purvalanol A or aminopurvalanol A for 30 mins, then allowed to adhere to cytochalasin D-retracted BAEC or BAEC-derived matrix for 30 min. Adhesion index is reported as the ratio of adhesion with inhibitors/adhesion without inhibitors of 6 replicate wells ±SD. B. Cdk inhibitors block Rb phosphorylation. Jurkat cells were treated with 20 μM aminopurvalanol A or purvanol A for 30 min, then lysed. Equal amounts of protein were separated by SDS-PAGE then blotted with a phospho-specific antibody to Cdk2 specific site on Rb (T821) (left) or Cdk4 specific site on Rb (249/252) (right).

FIG. 5. Cdk4 dominant negative (DN) construct inhibits LIA. A. Jurkat cells stably transfected with Cdk2 DN (left) or Cdk4 DN (right) or control vector were lysed and equal amounts of protein were separated by SDS-PAGE then blotted with an antibody to Cdk2 or Cdk4. B. Cdk2 and Cdk4 DN constructs specifically inhibit phosphorylation of Rb protein. Jurkat cell transfectants were lysed, equal amounts of protein were separated by SDS-PAGE then blotted with a phospho-specific antibody to Cdk 2 specific site on Rb (T821) (left) or Cdk 4 specific site on Rb (249/252) (right). C. Cdk4 DN, but not Cdk2 DN, inhibits ligand-induced adhesion. Control, Cdk2 dominant-negative and Cdk4 dominant-negative Jurkat cells were tested for unstimulated and phorbol ester-stimulated adhesion to BAEC matrix. Adhesion index is the adhesion of DN transfectants compared to adhesion of controls. Data are means of at least six independent experiments of five replicate wells each±SEM. D. Jurkat cell adhesion (DN or control) to different concentrations of fibronectin ±PDBu stimulation.

FIG. 6. Cdk4 siRNA inhibits LIA. Jurkat cells were transiently transfected with Cdk4 siRNA or control vector. After 48 hr, cells were allowed to adhere to high density fibronectin for 30 min (A). Concurrent Jurkat cell transfectants were lysed, equal amounts of protein were separated by SDS-PAGE then blotted with antibody to Cdk4 (B)

FIG. 7. Flow cytometry analysis of total β1, β1 activation epitope and α4 integrin surface expression in control and Cdk4DN Jurkat cells.

FIG. 8. Rb phosphorylation is not required for LIA. A. Jurkat cells stably transfected with DN Rb were allowed to adhere to low or high density FN with or without prior PDBu stimulation B. Lysates of Jurkat cells transfected with DN RB or control were blotted with antibody to total Rb (top) or phospho-specific-Rb (bottom).

FIG. 9. Transendothelial migration is inhibited by Cdk inhibitors. PBMC were allowed to adhere to confluent HUVEC monolayer for 30 min, then treated with Cdk inhibitor purvalanol. Cells that migrated through transwell filter after 3.5 h were counted. Each experiment was done in triplicate. Representative experiment is shown.

### DETAILED DESCRIPTION

### General Introduction and Overview

A method for preventing or treating inflammatory-related diseases in a mammalian subject is provided comprising administering to the subject one or more modulators of cyclin dependent kinase 4 (Cdk4) activity in an amount effective to reduce or eliminate the inflammatory-related disease or disorder or prevent its occurrence or recurrence. The target of the modulator is cyclin dependent kinase 4 (Cdk4). The modulator can be an inhibitor and an inhibitor can be a small chemical compound, peptide or peptide deriviative, siRNA, ribozyme, antisense, or antibody.

Leukocyte trafficking is a tightly regulated process essential for an appropriate inflammatory response. A new pathway is described herein that allows lymphocytes to adhere to exposed endothelial matrix in the absence of exogenous cytokine or chemokine stimulation. This ligand-induced adhesion (LIA) is integrin-mediated, but in contrast to phorbol ester stimulated adhesion, it is not dependent on the small GTPase Rap-1 activity. Instead, this novel pathway is dependent on cyclin dependent kinase (Cdk) activity, specifically Cdk4. The results described in the Exemplary Embodiments illustrate a previously unrecognized link between cell cycle and adhesion. This pathway can contribute to leukocyte trafficking in disorders characterized by abnormal leukocyte infiltration. In addition, it suggests that under certain conditions, cell proliferation/cell cycle could control leukocyte adhesion.

It is to be understood that this invention is not limited to particular methods, reagents, compounds compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a cell” includes a combination of two or more cells, and the like.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

“Cdk4,” as used herein, refers to cyclin dependent protein kinase 4. Cdk4 is a serine/threonine kinase. (the p21Cip1 protein is a potent stoichiometric inhibitor of cyclin-dependent kinase activity, Cyclin D1 activates cdk4). Cdk4 plays a major role in regulating cell cycle proliferation, and gene expression through phosphorylation of Retinoblastoma (Rb) protein. In addition, Cdk4 also regulates cell death, differentiation, and leukocyte adhesion regulation (as described herein).

“Kinase,” as used herein, refers to an enzyme which can catalyze the transfer of phosphate groups usually to a tyrosine, threonine or serine residue. Protein phosphorylation is a basic mechanism for modification of protein function in eukaryotic cells. Phosphorylation and dephophorylation of proteins is a chemical signaling mechanism used by cells to relay messages from the outside environment (i.e., outside the cell membrane) to the interior of the cell. Typically the signal is ultimately transferred to the nucleus where it alters gene expression. Tyrosine kinases are involved in immune, endocrine and nervous system physiology and pathology.

“Inflammatory-related disease or disorder” refers any condition characterized by inflammation at a site of injury or infection and includes, for example, autoimmune diseases, certain forms of infectious inflammatory states, undesirable leukocyte activity characteristic of organ transplants or other implants and virtually any other condition characterized by unwanted leukocyte activation.

The term “immune-mediated” refers to a process that is either autoimmune or inflammatory in nature.

“Nucleic acid molecule” includes DNA molecules (e.g., a cDNA or genomic DNA) and RNA molecules (e.g., an mRNA) and analogs of the DNA or RNA generated, e.g., by the use of nucleotide analogs. The nucleic acid molecule can be single-stranded or double-stranded, but preferably is double-stranded DNA. “Isolated or purified nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A “naturally-occurring” nucleic acid molecule refers to an RNA or DNA molecule having a nucleotide sequence that occurs in nature (e.g., encodes a natural protein).

“Gene” and “recombinant gene” refer to nucleic acid molecules which include an open reading frame encoding proteins, and can further include non-coding regulatory sequences, and introns.

An “isolated” or “purified” polypeptide or protein is substantially free of cellular material or other contaminating proteins from the cell or tissue source from which the protein is derived, or substantially free from chemical precursors or other chemicals when chemically synthesized. In one aspect, the language “substantially free” means a preparation of a protein of the invention having less than about 30%, 20%, 10% and more preferably 5% (by dry weight), of non-proteins of the invention (also referred to herein as a “contaminating protein”), or of chemical precursors or non-chemicals. When the protein, or biologically active portion thereof, is recombinantly produced, it is also preferably substantially free of culture medium, i.e., culture medium represents less than about 20%, more preferably less than about 10%, and most preferably less than about 5% of the volume of the protein preparation. The invention includes isolated or purified preparations of at least 0.01, 0.1, 1.0, and 10 milligrams in dry weight.

A “non-essential” amino acid residue is a residue that can be altered from the wild-type sequence of the protein sequences, without substantially altering a biological activity, whereas an “essential” amino acid residue results in such a change. For example, amino acid residues that are conserved among the polypeptides of the present invention, are predicted to be particularly not amenable to alteration.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a protein is preferably replaced with another amino acid residue from the same side chain family. Alternatively, in another aspect, mutations can be introduced randomly along all or part of a coding sequence, such as by saturation mutagenesis, and the resultant mutants can be screened for biological activity to identify mutants that retain activity. Following mutagenesis of the protein sequences, the encoded protein can be expressed recombinantly and the activity of the protein can be determined.

A “biologically active portion” of a protein includes a fragment of a protein which participates in an interaction between a molecule and an effector molecule. Biologically active portions of a protein include peptides comprising amino acid sequences sufficiently identical to or derived from the amino acid sequence of the protein. Typically, biologically active portions comprise a domain or motif with at least one activity of the protein of interest.

A biologically active portion of a protein can be a polypeptide which is, for example, 10, 25, 50, 100, 200, or more, amino acids in length. Biologically active portions of a protein can be used as targets for developing agents which modulate a Cdk4-mediated activity as described herein.

Calculations of homology or sequence identity (the terms are used interchangeably herein) between sequences are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred aspect, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred aspect, the percent identity between two amino acid sequences is determined using the (Needleman and Wunsch, J. Mol. Biol 48: 444-453, 1970) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred aspect, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a Blossum 62 scoring matrix with a gap penalty of 12, a gap extend penalty of 4, and a frameshift gap penalty of 5.

The percent identity between two amino acid or nucleotide sequences can be determined using the algorithm of (Meyers and Miller, CABIOS 4: 11-17, 1989) which has been incorporated into the ALIGN program (version 2.0), using a PAM 120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of (Altschul et al., J. Mol. Biol 215: 403-10, 1990). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in (Altschul et al., Nucleic Acids Res 25: 3389-3402, 1997). When utilizing BLAST and Gapped BLAST programs, the default parameters of the respective programs (e.g., XBLAST and NBLAST) can be used. See http://www.ncbi.nlm.nih.gov.

Particular polypeptides of the present invention have an amino acid sequence sufficiently identical or substantially identical to the amino acid sequence of the protein sequences. “Sufficiently identical” or “substantially identical” is used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain or common functional activity. For example, amino acid or nucleotide sequences that contain a common structural domain having at least about 60%, or 65% identity, likely 75% identity, more likely 85%, 90%, 91%, 92%, 93%, 94%, 95%, 96%, 97%, 98% or 99% identity are defined herein as sufficiently or substantially identical.

“Misexpression or aberrant expression”, as used herein, refers to a non-wild type pattern of gene expression, at the RNA or protein level. It includes: expression at non-wild type levels, i.e., over- or under-expression; a pattern of expression that differs from wild type in terms of the time or stage at which the gene is expressed, e.g., increased or decreased expression (as compared with wild type) at a predetermined developmental period or stage; a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus.

### Patient Population

Methods are provided for treating inflammatory-related diseases or disorders in a mammalian subject comprising administering to the subject one or more modulators of cyclin dependent kinase 4 (Cdk4) activity in an amount effective to reduce or eliminate the inflammatory-related disease or disorder or prevent its occurrence or recurrence.

“Subject” refers to a mammal, e.g., a human, or to an experimental or animal or disease model. The subject can also be a non-human animal, e.g., a horse, cow, goat, or other domestic animal. In one aspect, the methods of the invention are useful in treating, managing, or preventing diseases or disorders in patients that have been refractory or resistant to single agents capable of targeting a single specific kinase or kinase pathway. It is also envisioned that the methods of the invention are useful in treating, managing, or preventing diseases or disorders in patients that have undergone, are currently undergoing, or may in the future undergo other treatments. For example, the agents of the invention may be administered to individuals that have been administered other therapeutic agents, or that have undergone other therapies such as radiation or surgery.

The methods the invention are useful in untreated patients. The methods are also useful in the treatment of patients partially or completely refractory to current standard and experimental therapies. In one aspect, the invention provides therapeutic and prophylactic methods for the treatment or prevention of a disease, condition or disorder that has been shown to be or may be refractory or non-responsive to therapies other than those comprising administration of an agent capable of targeting one or more kinase or kinase pathway. In an alternate aspect, the invention provides therapeutic and prophylactic methods for the treatment or prevention of a disease, condition or disorder that has been shown to be or may be refractory or non-responsive to therapies comprising administration an agent capable of targeting a kinase or kinase pathway.

In another aspect, the methods of the invention are useful in treating, managing, or preventing diseases or disorders in patients that are currently receiving other therapies, such as, for example, anti-cancer therapies (e.g., chemotherapy, surgery, radiation therapy, antibody therapy, and the like); anti-inflammatory therapy (e.g., steroidal or non-steroidal anti-inflammatory drugs, antibody therapy, agonists, cytokine therapy, and the like).

In yet another aspect, the methods of the invention are useful in treating, managing, or preventing diseases or disorders in patients that have been determined to be pre-disposed to any disease condition, particularly to inflammatory-related diseases or disorders, or any of the diseases and disorders recited below.

### Diseases and Disorders

The present invention encompasses therapies which involve administering one or more agents to an animal, preferably a mammal, and most preferably a human, for preventing, treating, or ameliorating symptoms associated with a disease, disorder, or infection, associated with the modulating Cdk4 kinase activity. In one aspect, the invention relates to the prevention, treatment or amelioration of symptoms associated with a disease, disorder or infection, associated with the abnormal activity (e.g., abnormal upregulation or downregulation) of at least one cyclin dependent kinase, such as Cdk4. In some aspects, the methods of the invention are used in combination with one or more therapies such as, but not limited to, chemotherapies, radiation therapies, hormonal therapies, and/or biological therapies/immunotherapies.

The methods of the invention are useful in treating, preventing, managing or ameliorating a variety of diseases or disorders related to protein kinase activity, including, but not limited to, disorders related to the following: gene expression, cytoskeletal integrity, cell adhesion, cell cycle progression, differentiation and metabolism. Such diseases are controlled by the complex interplay of protein kinases and phosphatases, and associated malfunctions of cellular signaling have been linked to many diseases including cancer and diabetes. Sridhar et al., Pharmaceutical Research 17: 1345-1353, 2000. Therapeutic strategies that target protein kinases and therefore regulate signal transduction have become the subject of intense research. For example, protein kinase C and tyrosine kinases have been implicated in certain types of cancer, diabetes and complications associated with diabetes. In addition protein kinase C isoforms have been implicated in cellular changes observed in the vascular complications of diabetes. Sridhar et al., Pharmaceutical Research 17: 1345-1353, 2000; Chalfant et al., Mol. Endocrinol. 10: 1273-1281, 1996. As a result of the complexity associated with protein kinase pathways, overlap exists between various protein kinases and a wide range of diseases or disorders. The methods of the invention relate to the treatment, management, prevention or amelioration of diseases associated with a protein kinase including, but not limited to, cancer, inflammatory disorders, diabetes, obesity, angiogenesis disorders and cardiovascular disorders.

In particular, the methods of the invention are useful for the prevention, treatment, management and/or amelioration of various diseases. By way of example, and not meant to limit, examples of the types of classes of disease that can be prevented, treated or managed include, but are not limited to, inflammatory conditions. Inflammatory-related diseases and disorders include, but not limited to: diabetes (such as Type II diabetes, Type I diabetes, diabetes insipidus, diabetes mellitus, maturity-onset diabetes, juvenile diabetes, insulin-dependant diabetes, non-insulin dependant diabetes, malnutrition-related diabetes, ketosis-prone diabetes or ketosis-resistant diabetes); nephropathy (such as glomerulonephritis or acute/chronic kidney failure); obesity (such as hereditary obesity, dietary obesity, hormone related obesity or obesity related to the administration of medication); hearing loss (such as that from otitis extema or acute otitis media); fibrosis related diseases (such as pulmonary interstitial fibrosis, renal fibrosis, cystic fibrosis, liver fibrosis, wound-healing or burn-healing, wherein the burn is a first-, second- or third-degree burn and/or a thermal, chemical or electrical burn); arthritis (such as rheumatoid arthritis, rheumatoid spondylitis, osteoarthritis or gout); an allergy; allergic rhinitis; acute respiratory distress syndrome; asthma; bronchitis; an inflammatory bowel disease (such as irritable bowel syndrome, mucous colitis, ulcerative colitis, Crohn's disease, gastritis, esophagitis, pancreatitis or peritonitis); or an autoimmune disease (such as scleroderma, systemic lupus erythematosus, myasthenia gravis, transplant rejection, endotoxin shock, sepsis, psoriasis, eczema, dermatitis or multiple sclerosis).

In one particular aspect, the inflammatory-related disease or disorder is diabetes, nephropathy, obesity, hearing loss, fibrosis related disease, arthritis, allergy, allergic rhinitis, acute respiratory distress syndrome, asthma, bronchitis, inflammatory bowel disease, an autoimmune disease, hepatitis, atopic dermatitis, pemphigus, glomerulonephritis, atherosclerosis, sarcoidosis, ankylosing spondylitis, Wegner's syndrome, Goodpasture's syndrome, giant cell arteritis, polyarteritis nodosa, idiopathic pulmonary fibrosis, acute lung injury, chronic obstructive pulmonary disease, post-influenza pneumonia, SARS, tuberculosis, malaria, sepsis, cerebral malaria, Chagas disease, schistosomiasis, bacterial and viral meningitis, cystic fibrosis, multiple sclerosis, Alzheimer's disease, encephalomyelitis, sickle cell anemia, pancreatitis, transplantation, systemic lupus erythematosis, thyroiditis, and radiation pneumonitis, lymphocytosis syndrome, or lymphocytic interstitial pneumonitis.

A. Inflammatory Disorders, Diabetes and Obesity

As described earlier, kinase activity has been associated with inflammatory disorders as well as with diabetes and its related effects. Indeed, a number of kinases have been implicated in cellular changes observed in the vascular complications of diabetes. Chalfant et al., Mol. Endocrinol 10: 1273-1281, 1996. Furthermore, as obesity is closely related with the insulin resistance found in type II diabetes, kinases that interfere with insulin action have been implicated in disorders related to obesity as well as diabetes. Hirosumi et al., Nature 420: 333-336, 2002. Obesity and diabetes are also closely associated with the chronic inflammatory response that is mediated by kinases in various signal cascades. Hirosumi et al., Nature 420: 333-336, 2002. As such, the methods of the invention relate to the management, treatment or prevention of inflammatory disorders, diabetes, obesity and their associated pathologies. Obesity treated according to the methods of the invention include hereditary obesity, dietary obesity, obesity related to the administration of medication or a course of therapy, obesity associated with diabetes, Examples of inflammatory and diabetes related disorders that may be managed, treated, prevented or ameliorated using the methods of the invention, include, but are not limited to, type I diabetes, juvenile diabetes, diabetes mellitus type II (NIDDM), noninsulin-dependent diabetes mellitus, maturity-onset diabetes dystrophia myotonica, malnutrition-related diabetes, ketosis-prone diabetes, ketoresistant diabetes, myotonic dystrophy 1, Steinert disease, liver glycogenosis, x-linked type I, hepatic phosphorylase kinase deficiency, phosphorylase kinase deficiency of liver, glycogenosis VIIIA, x-linked liver glycogenosis, phosphorylase kinase, liver glycogenosis x-linked type II, glycogen storage disease IX, glycogen storage disease VIII, lipoprotein lipase deficiency, 1pl deficiency, familial hyperchylomicronemia, hyperlipemia burger-grutz type, essential familial hyperlipemia, lipase D deficiency, type IA hyperlipoproteinemia, familial chylomicronemia, type I GM2-gangliosidosis, B variant GM2 gangliosidosis, hexosaminidase A deficiency, Tay-Sachs disease, pseudo-AB variant Tay-Sachs disease, mucoviscidosis, cystic fibrosis, galactose-1-phosphate uridylyltransferase deficiency, galt deficiency, classic galactosemia, chronic granulomatous disease, type I autosomal cytochrome-b-positive granulomatous disease, deficiency of neutrophil cytosol factor 1, deficiency of soluble oxidase component II, deficiency of soc2, deficiency of p47-phox, type I Gaucher disease, noncerebral juvenile gaucher disease, glucocerebrosidase deficiency, acid beta-glucosidase deficiency, hereditary sideroblastic anemia, hereditary iron-loading anemia, chronic granulomatous disease, X-linked cytochrome-b-negative granulomatous disease, asthma, encephalitis, inflammatory bowel disease, chronic obstructive pulmonary disease (COPD), allergic disorders, septic shock, pulmonary fibrosis, undifferentiated spondyloarthropathy, undifferentiated arthropathy, arthritis, inflammatory osteolysis, chronic inflammation resulting from chronic viral or bacteria infections, asthma, encephilitis, inflammatory bowel disease, chronic obstructive pulmonary disease (COPD), allergic disorders, septic shock, pulmonary fibrosis, undifferentiated spondyloarthropathy, undifferentiated arthropathy, arthritis, inflammatory osteolysis, and chronic inflammation resulting from chronic viral or bacteria infections.

Many autoimmune disorders are associated with inflammatory conditions. Thus, there is overlap between what is considered an autoimmune disorder and an inflammatory disorder. Therefore, some-autoimmune disorders may also be characterized as inflammatory disorders. Examples of autoimmune disorders that may be managed, treated or prevented using the methods of the invention include, but are not limited to, alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erythematosus, Meniere's disease, mixed connective tissue disease, multiple sclerosis, type 1 or immune-mediated diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, Sjogren's syndrome, stiff-man syndrome, systemic lupus erythematosus, lupus erythematosus, takayasu arteritis, temporal arteristis/giant cell arteritis, ulcerative colitis, uveitis, vasculitides such as dermatitis herpetiformis vasculitis, vitiligo, and Wegener's granulomatosis.

B. Diseases Involving Microorganisms Including Bacteria, Viruses and Fungi

The methods of the invention can be used to target kinases that are related to infections by a microorganisms. Such methods can be used to prevent infection of a host by a microorganism, such as, but not limited to, a bacteria, virus or fumgus. The methods of the invention can also be used to treat, prevent or ameliorate symptoms or conditions that are associated with infection by a microorganism, such as, but not limited to, a virus, bacteria or a fungus. Microorganisms, including viruses, that can infect an organism and that rely upon kinases for transmission, survival or homeostasis are known in the art. Such infectious agents that can be treated, prevented, managed or ameliorated using the methods of the invention include, but are not limited to, bacteria (e.g., gram positive bacteria, gram negative bacteria, aerobic bacteria, Spirochetes, Mycobacteria, Rickettsias, Chlamydias, and the like), parasites, fungi (e.g., Candida albicans, Aspergillus, and the like), viruses (e.g., DNA viruses, RNA viruses, and the like), or tumors. Viral infections that can be treated, prevented, managed or ameliorated include, but are not limited to, human immunodeficiency virus (HIV); hepatitis A virus, hepatitis B virus, hepatitis C virus, hepatitis D virus, or other hepatitis viruses; cytomegalovirus, herpes simplex virus-1 (-2, -3,-4,-5,-6), human papilloma viruses; Respiratory syncytial virus (RSV), Parainfluenza virus (PIV), Epstein Barr virus, Metapneumovirus (MPV) or any other viral infections.

### Cyclin Dependent Kinase (CDK)

In one aspect, the kinases that are targeted using the methods of the invention include kinases that are cyclin dependent or from the CDK family of kinases. While the invention embodies the modulation of any member of the CDK family of kinases, in a particular aspect, the Cdk is Cdk4.

Cdks are Ser/Thr kinases that regulate progression through the cell cycle. However, there is increasing evidence that Cdks, as well as cyclins and Cdk inhibitors (CKIs) are important for other functions, including cytoskeleton rearrangement and cell migration. Besson et al., Nat Rev Cancer 4: 948-955, 2004. Cdk5 plays an important role in neuronal cell function and can phosphorylate focal adhesion kinase (FAK) and contribute to neurite outgrowth and migration. Gao et al, Mol Cancer Res 1: 12-24, 2002; Negash et al., J Cell Sci 115: 2109-2117, 2002. Other Cdks including Cdk 1, 4, 6 regulate neuronal cell death. A recent report described a novel role for Cdk1 (cdc2) as a downstream effector of the integrin αvβ3, leading to cell migration. Manes et al., J Cell Biol 161: 817-826, 2003. Furthermore, Cdk1 localized to membrane ruffles of migrating cells. Manes et al., J Cell Biol 161: 817-826, 2003. Thus, there is increasing evidence that Cdks may have non-traditional roles in various cell behaviors, including those related to adhesion and migration.

Cdk4 along with Cdk6 act early in the cell cycle and are involved in the transition from G1 to S phase. When G0 cells are stimulated to divide (mitogens, and the like), Cdk4 is activated by phosphorylation at threonine-172 (by Cdk activating kinase, CAK-composed of 3 subunits: Cdk7, cyclin H, MAT1) and binding to its cyclin counterpart (cyclin D). The cyclin D/Cdk 4 complex translocates to the nucleus where it phosphorylates target proteins and allows cell cycle progression. The major target for Cdk4 phosphorylation is retinoblastoma (Rb) protein. The hyperphosphorylation of Rb inactivates the protein, which results in the release of the transcription factors E2F and DP1. These transcription factors regulate the gene expression of proteins essential for G1/S phase transition and S1 progression. Until recently, the only known substrates for cyclinD/Cdk4 were Rb family members. However, recent work demonstrated that Smad3, a downstream effector of TGFβ, was a substrate for CyclinD/Cdk4. Matsuura, Nature 430: 226-231, 2004. Cdk4 phosphorylation of Smad3 (at sites distinct from TGFβ phosphorylation) blocked its anti-proliferative function. In Drosophila, Cdk4/cyclin D can also bind the STAT homologue, STAT92E, which increased STAT92E stability and activity. Chen et al., Dev Cell 4: 179-190, 2003. However, there was no evidence that Cdk4 phosphorylated STAT92E.

Cdks are also regulated by Cdk inhibitors: p16/INK members specifically inhibit Cdk4 and cdk6, while Cip/Kip members p21, p27, and p57 inhibit a broader spectrum of CDKs. Interestingly, the Cdk4 knockout mouse is viable, although most mice are sterile, with small body size. Other defects include abnormal pancreatic islet cell formation leading to insulin dependent diabetes and abnormalities in hypothalamic-pituitary axis. Rane et al., Nat Genet 22: 44-52, 1999; Sherr, Genes Dev 18: 2699-2711, 2004.

Cell adhesion and cell cycle. It is well accepted that cell adhesion can regulate cell cycle progression. In most anchorage-dependent cells, integrin adhesion is necessary for progression through the cell cycle, with loss of adhesion resulting in G1 phase cell cycle arrest. Furthermore, interactions between integrins and growth factor receptors further potentiates the effect on cell cycle progression. Schwartz and Assoian, J Cell Sci 114: 2553-2560, 2001; this reference is herein incorporated by reference in its entirety for all purposes. While it is clear that integrin adhesion regulates cell cycle, little is known about whether the cell cycle regulates integrin adhesion. As disclosed herein, data suggests a potential link in this regard as well.

### RNA and DNA Interference Methods

A. Short Interfering RNAs (RNAi)

RNA interference (RNAi) is a mechanism of post-transcriptional gene silencing mediated by double-stranded RNA (dsRNA), which is distinct from antisense and ribozyme-based approaches (see Jain, Pharmacogenomics 5: 239-42, 2004 for a review of RNAi and siRNA). RNA interference is useful in a method for treating a inflammatory-related disease or disorder in a mammal by administering to the mammal a nucleic acid molecule (e.g., dsRNA) that hybridizes under stringent conditions to a cyclin dependent kinase target gene, and attenuates expression of said target gene. dsRNA molecules are believed to direct sequence-specific degradation of mRNA in cells of various types after first undergoing processing by an RNase III-like enzyme called DICER (Bernstein et al., Nature 409: 363, 2001) into smaller dsRNA molecules comprised of two 21 nt strands, each of which has a 5′ phosphate group and a 3′ hydroxyl, and includes a 19 nt region precisely complementary with the other strand, so that there is a 19 nt duplex region flanked by 2 nt-3′ overhangs. RNAi is thus mediated by short interfering RNAs (siRNA), which typically comprise a double-stranded region approximately 19 nucleotides in length with 1-2 nucleotide 3′ overhangs on each strand, resulting in a total length of between approximately 21 and 23 nucleotides. In mammalian cells, dsRNA longer than approximately 30 nucleotides typically induces nonspecific mRNA degradation via the interferon response. However, the presence of siRNA in mammalian cells, rather than inducing the interferon response, results in sequence-specific gene silencing.

In general, a short, interfering RNA (siRNA) comprises an RNA duplex that is preferably approximately 19 basepairs long and optionally further comprises one or two single-stranded overhangs or loops. An siRNA may comprise two RNA strands hybridized together, or may alternatively comprise a single RNA strand that includes a self-hybridizing portion. siRNAs may include one or more free strand ends, which may include phosphate and/or hydroxyl groups. siRNAs typically include a portion that hybridizes under stringent conditions with a target transcript. One strand of the siRNA (or, the self-hybridizing portion of the siRNA) is typically precisely complementary with a region of the target transcript, meaning that the siRNA hybridizes to the target transcript without a single mismatch. In certain embodiments of the invention in which perfect complementarity is not achieved, it is generally preferred that any mismatches be located at or near the siRNA termini.

siRNAs have been shown to downregulate gene expression when transferred into mammalian cells by such methods as transfection, electroporation, or microinjection, or when expressed in cells viany of a variety of plasmid-based approaches. RNA interference using siRNA (is reviewed in, e.g., Tuschl, Nat. Biotechnol. 20: 446-448, 2002; See also Yu, J. et al., Proc. Natl. Acad. Sci., 99: 6047-6052, 2002; Sui et al., Proc. Natl. Acad. Sci USA. 99: 5515-5520, 2002; Paddison et al., Genes and Dev. 16: 948-958, 2002; Brummelkamp et al., Science 296: 550-553, 2002; Miyagashi et al., Nat. Biotech. 20: 497-500, 2002; Paul et al., Nat. Biotech. 20: 505-508, 2002). As described in these and other references, the siRNA may consist of two individual nucleic acid strands or of a single strand with a self-complementary region capable of forming a hairpin (stem-loop) structure. A number of variations in structure, length, number of mismatches, size of loop, identity of nucleotides in overhangs, etc., are consistent with effective siRNA-triggered gene silencing. While not wishing to be bound by any theory, it is thought that intracellular processing (e.g., by DICER) of a variety of different precursors results in production of siRNA capable of effectively mediating gene silencing. Generally it is preferred to target exons rather than introns, and it may also be preferable to select sequences complementary to regions within the 3′ portion of the target transcript. Generally it is preferred to select sequences that contain approximately equimolar ratio of the different nucleotides and to avoid stretches in which a single residue is repeated multiple times.

siRNAs may thus comprise RNA molecules having a double-stranded region approximately 19 nucleotides in length with 1-2 nucleotide 3′ overhangs on each strand, resulting in a total length of between approximately 21 and 23 nucleotides. As used herein, siRNAs also include various RNA structures that may be processed in vivo to generate such molecules. Such structures include RNA strands containing two complementary elements that hybridize to one another to form a stem, a loop, and optionally an overhang, preferably a 3′ overhang. Preferably, the stem is approximately 19 bp long, the loop is about 1-20, more preferably about 4-10, and most preferably about 6-8 nt long and/or the overhang is about 1-20, and more preferably about 2-15 nt long. In certain embodiments of the invention the stem is minimally 19 nucleotides in length and may be up to approximately 29 nucleotides in length. Loops of 4 nucleotides or greater are less likely subject to steric constraints than are shorter loops and therefore may be preferred. The overhang may include a 5′ phosphate and a 3′ hydroxyl. The overhang may but need not comprise a plurality of U residues, e.g., between 1 and 5 U residues. Classical siRNAs as described above trigger degradation of mRNAs to which they are targeted, thereby also reducing the rate of protein synthesis. In addition to siRNAs that act via the classical pathway, certain siRNAs that bind to the 3′ UTR of a template transcript may inhibit expression of a protein encoded by the template transcript by a mechanism related to but distinct from classic RNA interference, e.g., by reducing translation of the transcript rather than decreasing its stability. Such RNAs are referred to as microRNAs (mRNAs) and are typically between approximately 20 and 26 nucleotides in length, e.g., 22 nt in length. It is believed that they are derived from larger precursors known as small temporal RNAs (stRNAs) or mRNA precursors, which are typically approximately 70 nt long with an approximately 4-15 nt loop (see Grishok et al., Cell 106: 23-24, 2001; Hutvagner et al., Science 293: 834-838, 2001; Ketting et al., Genes Dev., 15: 2654-2659, 2001). Endogenous RNAs of this type have been identified in a number of organisms including mammals, suggesting that this mechanism of post-transcriptional gene silencing may be widespread. Lagos-Quintana et al., Science 294: 853-858, 2001; Pasquinelli, Trends in Genetics 18: 171-173, 2002, and references in the foregoing two articles. MicroRNAs have been shown to block translation of target transcripts containing target sites in mammalian cells. Zeng et al., Molecular Cell 9: 1-20, 2002.

siRNAs such as naturally occurring or artificial (i.e., designed by humans) mRNAs that bind within the 3′ UTR (or elsewhere in a target transcript) and inhibit translation may tolerate a larger number of mismatches in the siRNA/template duplex, and particularly may tolerate mismatches within the central region of the duplex. In fact, there is evidence that some mismatches may be desirable or required as naturally occurring stRNAs frequently exhibit such mismatches as do mRNAs that have been shown to inhibit translation in vitro. For example, when hybridized with the target transcript such siRNAs frequently include two stretches of perfect complementarity separated by a region of mismatch. A variety of structures are possible. For example, the mRNA may include multiple areas of nonidentity (mismatch). The areas of nonidentity (mismatch) need not be symmetrical in the sense that both the target and the mRNA include nonpaired nucleotides. Typically the stretches of perfect complementarity are at least 5 nucleotides in length, e.g., 6, 7, or more nucleotides in length, while the regions of mismatch may be, for example, 1, 2, 3, or 4 nucleotides in length.

Hairpin structures designed to mimic siRNAs and mRNA precursors are processed intracellularly into molecules capable of reducing or inhibiting expression of target transcripts. McManus et al., RNA 8: 842-850, 2002. These hairpin structures, which are based on classical siRNAs consisting of two RNA strands forming a 19 bp duplex structure are classified as class I or class II hairpins. Class I hairpins incorporate a loop at the 5′ or 3′ end of the antisense siRNA strand (i.e., the strand complementary to the target transcript whose inhibition is desired) but are otherwise identical to classical siRNAs. Class II hairpins resemble mRNA precursors in that they include a 19 nt duplex region and a loop at either the 3′ or 5′ end of the antisense strand of the duplex in addition to one or more nucleotide mismatches in the stem. These molecules are processed intracellularly into small RNA duplex structures capable of mediating silencing. They appear to exert their effects through degradation of the target mRNA rather than through translational repression as is thought to be the case for naturally occurring mRNAs and stRNAs.

Thus it is evident that a diverse set of RNA molecules containing duplex structures is able to mediate silencing through various mechanisms. For the purposes of the present invention, any such RNA, one portion of which binds to a target transcript and reduces its expression, whether by triggering degradation, by inhibiting translation, or by other means, is considered to be an siRNA, and any structure that generates such an siRNA (i.e., serves as a precursor to the RNA) is useful in the practice of the present invention.

In the context of the present invention, siRNAs are useful both for therapeutic purposes, e.g., to modulate the expression of a cyclin dependent kinase protein in a subject at risk of or suffering from an inflammatory-related disease or disorder and for various of the inventive methods for the identification of compounds for treatment of an inflammatory-related disease or disorder that modulate the activity or level of the molecules described herein. In a preferred aspect, the therapeutic treatment of an inflammatory-related disease or disorder, with an antibody, antisense vector, peptide, or double stranded RNA vector.

The invention therefore provides a method of inhibiting expression of a gene encoding a cyclin dependent kinase protein comprising the step of (i) providing a biological system in which expression of a gene encoding cyclin dependent kinase protein is to be inhibited; and (ii) contacting the system with an siRNA targeted to a transcript encoding the cyclin dependent kinase protein. According to certain embodiments of the invention the cyclin dependent kinase protein is encoded by a gene within or linked to an inflammatory-related disease or disorder susceptibility locus, or within which a functional mutation causing or contributing to susceptibility or development of an inflammatory-related disease or disorder may exist. In other embodiments, cyclin dependent kinase proteins are inhibited. According to certain embodiments of the invention the biological system comprises a cell, and the contacting step comprises expressing the siRNA in the cell. According to certain embodiments of the invention the biological system comprises a subject, e.g., a mammalian subject such as a mouse or human, and the contacting step comprises administering the siRNA to the subject or comprises expressing the siRNA in the subject. According to certain embodiments of the invention the siRNA is expressed inducibly and/or in a cell-type or tissue specific manner.

By “biological system” is meant any vessel, well, or container in which biomolecules (e.g., nucleic acids, polypeptides, polysaccharides, lipids, and the like) are placed; a cell or population of cells; a tissue; an organ; an organism, and the like. Typically the biological system is a cell or population of cells, but the method can also be performed in a vessel using purified or recombinant proteins.

The invention provides siRNA molecules targeted to a transcript encoding any cyclin dependent kinase protein. In particular, the invention provides siRNA molecules selectively or specifically targeted to a transcript encoding a polymorphic variant of such a transcript, wherein existence of the polymorphic variant in a subject is indicative of susceptibility to or presence of an inflammatory-related disease or disorder. The terms “selectively” or “specifically targeted to”, in this context, are intended to indicate that the siRNA causes greater reduction in expression of the variant than of other variants (i.e., variants whose existence in a subject is not indicative of susceptibility to or presence of an inflammatory-related disease or disorder). The siRNA, or collections of siRNAs, may be provided in the form of kits with additional components as appropriate.

B. Short Hairpin RNAs (shRNA)

RNA interference (RNAi), a mechanism of post-transcriptional gene silencing mediated by double-stranded RNA (dsRNA), is useful in a method for treating an inflammatory-related disease or disorder in a mammal by administering to the mammal a nucleic acid molecule (e.g., dsRNA) that hybridizes under stringent conditions to a cyclin dependent kinase target gene, and attenuates expression of said target gene (see Jain, Pharmacogenomics 5: 239-42, 2004 for a review of RNAi and siRNA). A further method of RNA interference in the present invention is the use of short hairpin RNAs (shRNA). A plasmid containing a DNA sequence encoding for a particular desired siRNA sequence is delivered into a target cell via transfection or virally-mediated infection. Once in the cell, the DNA sequence is continuously transcribed into RNA molecules that loop back on themselves and form hairpin structures through intramolecular base pairing. These hairpin structures, once processed by the cell, are equivalent to transfected siRNA molecules and are used by the cell to mediate RNAi of the desired protein. The use of shRNA has an advantage over siRNA transfection as the former can lead to stable, long-term inhibition of protein expression. Inhibition of protein expression by transfected siRNAs is a transient phenomenon that does not occur for times periods longer than several days. In some cases, this may be preferable and desired. In cases where longer periods of protein inhibition are necessary, shRNA mediated inhibition is preferable.

C. Full and Partial Length Antisense RNA Transcripts

Antisense RNA transcripts have a base sequence complementary to part or all of any other RNA transcript in the same cell. Such transcripts have been shown to modulate gene expression through a variety of mechanisms including the modulation of RNA splicing, the modulation of RNA transport and the modulation of the translation of mRNA. Denhardt, Ann N Y Acad. Sci. 660: 70, 1992; Nellen, Trends Biochem. Sci. 18: 419, 1993; Baker et al, Biochim. Biophys. Acta, 1489: 3, 1999; Xu et al., Gene Therapy 7: 438, 2000; French et al., Curr. Opin. Microbiol. 3: 159, 2000; Terryn et al., Trends Plant Sci. 5: 1360, 2000.

D. Antisense RNAnd DNA Oligonucleotides

Antisense nucleic acids are generally single-stranded nucleic acids (DNA, RNA, modified DNA, or modified RNA) complementary to a portion of a target nucleic acid (e.g., an mRNA transcript) and therefore able to bind to the target to form a duplex. Typically they are oligonucleotides that range from 15 to 35 nucleotides in length but may range from 10 up to approximately 50 nucleotides in length. Binding typically reduces or inhibits the function of the target nucleic acid. For example, antisense oligonucleotides may block transcription when bound to genomic DNA, inhibit translation when bound to mRNA, and/or lead to degradation of the nucleic acid. Reduction in expression of a cyclin dependent kinase polypeptide may be achieved by the administration of antisense nucleic acids or peptide nucleic acids comprising sequences complementary to those of the mRNA that encodes the polypeptide. Antisense technology and its applications are well known in the art and are described in Phillips, M. I. (ed.) Antisense Technology, Methods Enzymol., 2000, Volumes 313 and 314, Academic Press, San Diego, and references mentioned therein (see also Crooke, S. (ed.) ANTISENSE DRUG TECHNOLOGY: PRINCIPLES, STRATEGIES, AND APPLICATIONS (1^{st} Edition) Marcel Dekker; and references cited therein).

Antisense oligonucleotides can be synthesized with a base sequence that is complementary to a portion of any RNA transcript in the cell. Antisense oligonucleotides may modulate gene expression through a variety of mechanisms including the modulation of RNA splicing, the modulation of RNA transport and the modulation of the translation of mRNA (Denhardt, Ann N Y Acad Sci. 660: 70-6, 1992). Various properties of antisense oligonucleotides including stability, toxicity, tissue distribution, and cellular uptake and binding affinity may be altered through chemical modifications including (i) replacement of the phosphodiester backbone (e.g., peptide nucleic acid, phosphorothioate oligonucleotides, and phosphoramidate oligonucleotides), (ii) modification of the sugar base (e.g., 2′-O-propylribose and 2′-methoxyethoxyribose), and (iii) modification of the nucleoside (e.g., C-5 propynyl U, C-5 thiazole U, and phenoxazine C). Wagner, Nat. Medicine 1: 1116, 1995; Varga et al., Immun. Lett. 69: 217, 1999; Neilsen, Curr. Opin. Biotech. 10: 71, 1999; Woolf, Nucleic Acids Res. 18: 1763, 1990.

The invention provides a method of inhibiting expression of a gene encoding a cyclin dependent kinase protein comprising the step of (i) providing a biological system in which expression of a gene encoding a cyclin dependent kinase protein is to be inhibited; and (ii) contacting the system with an antisense molecule that hybridizes to a transcript encoding the cyclin dependent kinase protein. According to certain embodiments of the invention the cyclin dependent kinase protein is encoded by a gene within or linked to an inflammatory-related disease or disorder susceptibility locus, or within which a functional mutation causing or contributing to an inflammatory-related disease or disorder or development of an inflammatory-related disease or disorder may exist. In other embodiments, cyclin dependent kinase proteins are inhibited. According to certain embodiments of the invention the biological system comprises a cell, and the contacting step comprises expressing the antisense molecule in the cell. According to certain embodiments of the invention the biological system comprises a subject, e.g., a mammalian subject such as a mouse or human, and the contacting step comprises administering the antisense molecule to the subject or comprises expressing the antisense molecule in the subject. The expression may be inducible and/or tissue or cell type-specific. The antisense molecule may be an oligonucleotide or a longer nucleic acid molecule. The invention provides such antisense molecules.

E. Ribozymes

Certain nucleic acid molecules referred to as ribozymes or deoxyribozymes have been shown to catalyze the sequence-specific cleavage of RNA molecules. The cleavage site is determined by complementary pairing of nucleotides in the RNA or DNA enzyme with nucleotides in the target RNA. Thus, RNAnd DNA enzymes can be designed to cleave to any RNA molecule, thereby increasing its rate of degradation. Cotten et al, EMBO J. 8: 3861-3866, 1989; Usman et al., Nucl. Acids Mol. Biol. 10: 243, 1996; Usman et al., Curr. Opin. Struct. Biol. 1: 527, 1996; Sun et al., Pharmacol. Rev., 52: 325, 2000. See also e.g., Cotten et al, EMBO J. 8: 3861-3866, 1989.

The invention provides a method of inhibiting expression of a gene encoding a cyclin dependent kinase protein comprising the step of (i) providing a biological system in which expression of a gene encoding a cyclin dependent kinase protein is to be inhibited; and (ii) contacting the system with a ribozyme that hybridizes to a transcript encoding the cyclin dependent kinase protein and directs cleavage of the transcript. According to certain embodiments of the invention the cyclin dependent kinase protein is encoded by a gene within or linked to an inflammatory-related disease or disorder susceptibility locus, or within which a functional mutation causing or contributing to susceptibility or development of an inflammatory-related disease or disorder may exist. In other embodiments, cyclin dependent kinase proteins are inhibited. According to certain embodiments of the invention the biological system comprises a cell, and the contacting step comprises expressing the ribozyme in the cell. According to certain embodiments of the invention the biological system comprises a subject, e.g., a mammalian subject such as a mouse or human, and the contacting step comprises administering the ribozyme to the subject or comprises expressing the ribozyme in the subject. The expression may be inducible and/or tissue or cell-type specific according to certain embodiments of the invention. The invention provides ribozymes designed to cleave transcripts encoding cyclin dependent kinase proteins, or polymorphic variants thereof, as described above.

### CDK Inhibitors

The present invention provides a method for preventing or treating inflammatory-related diseases in a mammalian subject is provided comprising administering to the subject one or more modulators of cyclin dependent kinase 4 (Cdk4) activity in an amount effective to reduce or eliminate the inflammatory-related disease or disorder or prevent its occurrence or recurrence.

“Immune cell response” refers to the response of immune system cells to external or internal stimuli (e.g., antigen, cytokines, chemokines, and other cells) producing biochemical changes in the immune cells that result in immune cell migration, killing of target cells, phagocytosis, production of antibodies, other soluble effectors of the immune response, and the like.

“Leukocytes” as used herein has the normal meaning in the art, and refers to β1 integrin receptors expressing cells including T and B lymphocytes, monocytes, dendritic cells, eosinophils cells found in the blood, lymph, and lymphoid tissues.

“T lymphocyte response” and “T lymphocyte activity” are used here interchangeably to refer to the component of immune response dependent on T lymphocytes (i.e., the proliferation and/or differentiation of T lymphocytes into helper, cytotoxic killer, or suppressor T lymphocytes, the provision of signals by helper T lymphocytes to B lymphocytes that cause or prevent antibody production, the killing of specific target cells by cytotoxic T lymphocytes, and the release of soluble factors such as cytokines that modulate the function of other immune cells).

“Monocytes” are large, circulating, phagocytic white blood cells, having a single well-defined nucleus and very fine granulation in the cytoplasm. Monocytes constitute from 3 to 8% of the white blood cells in humans. Monocytes later emigrate from blood into the tissues of the body and there differentiate into cells called macrophages which play an important role in killing of some bacteria, protozoa, and tumor cells, release substances that stimulate other cells of the immune system, and are involved in antigen presentation.

“Immune response” refers to the concerted action of leukocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues.

“Treating” or “treatment” includes the administration of the compositions, compounds or agents of the present invention to prevent or delay the onset of the symptoms, complications, or biochemical indicia of a disease, alleviating or ameliorating the symptoms or arresting or inhibiting further development of the disease, condition, or disorder (e.g., an inflammatory-related disease or disorder). “Treating” further refers to any indicia of success in the treatment or amelioration or prevention of the disease, condition, or disorder (e.g., an inflammatory-related disease or disorder), including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with an inflammatory-related disease or disorder. The term “therapeutic effect” refers to the reduction, elimination, or prevention of the disease, symptoms of the disease, or side effects of the disease in the subject. “Treating” or “treatment” using the methods of the present invention includes preventing the onset of symptoms in a subject that can be at increased risk of an inflammatory-related disease or disorder but does not yet experience or exhibit symptoms, inhibiting the symptoms of an inflammatory-related disease or disorder (slowing or arresting its development), providing relief from the symptoms or side-effects of an inflammatory-related disease or disorder (including palliative treatment), and relieving the symptoms of an inflammatory-related disease or disorder (causing regression). Treatment can be prophylactic (to prevent or delay the onset of the disease, or to prevent the manifestation of clinical or subclinical symptoms thereof) or therapeutic suppression or alleviation of symptoms after the manifestation of the disease or condition.

The ability of a molecule to bind to cyclin dependent kinase can be determined, for example, by the ability of the putative ligand to bind to cyclin dependent kinase immunoadhesin coated on an assay plate. Specificity of binding can be determined by comparing binding to non-cyclin dependent kinase immunoadhesin.

In one aspect, antibody binding to cyclin dependent kinase can be assayed by either immobilizing the ligand or the receptor. For example, the assay can include immobilizing cyclin dependent kinase fused to a His tag onto Ni-activated NTA resin beads. Antibody can be added in an appropriate buffer and the beads incubated for a period of time at a given temperature. After washes to remove unbound material, the bound protein can be released with, for example, imidazol, SDS, buffers with a high pH, and the like and analyzed.

“Modulator” includes inhibitors and activators. Inhibitors are agents that, e.g., bind to, partially or totally block stimulation, decrease, prevent, delay activation, inactivate, desensitize, or down regulate the activity of cyclin dependent kinase, e.g., antagonists. Activators are agents that, e.g., bind to, stimulate, increase, open, activate, facilitate, enhance activation, sensitize or up regulate the activity of cyclin dependent kinase, e.g., agonists. Modulators include agents that, e.g., alter the interaction of cyclin dependent kinase with proteins that bind activators or inhibitors, receptors, including proteins, peptides, lipids, carbohydrates, polysaccharides, or combinations of the above, e.g., lipoproteins, glycoproteins, and the like. Modulators include genetically modified versions of naturally-occurring cyclin dependent kinase ligands, e.g., with altered activity, as well as naturally occurring and synthetic ligands, antagonists, agonists, small chemical molecules and the like. Such assays for inhibitors and activators include, e.g., applying putative modulator compounds to a cell expressing a cyclin dependent kinase and then determining the functional effects on cyclin dependent kinase activity, as described herein. Samples or assays comprising cyclin dependent kinase that are treated with a potential activator, inhibitor, or modulator are compared to control samples without the inhibitor, activator, or modulator to examine the extent of inhibition. Control samples (untreated with inhibitors) can be assigned a relative cyclin dependent kinase is activity value of 100%. Inhibition of cyclin dependent kinase is achieved when the cyclin dependent kinase activity value relative to the control is about 80%, optionally 50% or 25-0%. Activation of cyclin dependent kinase is achieved when the cyclin dependent kinase activity value relative to the control is 110%, optionally 150%, optionally 200-500%, or 1000-3000% higher.

“Inhibitors,” “activators,” and “modulators” of cyclin dependent kinase protein, e.g., Cdk4, activity are used to refer to inhibitory, activating, or modulating molecules, respectively, identified using in vitro and in vivo assays for cyclin dependent kinase activity, e.g., ligands, binding partners, agonists, antagonists, and their homologs and mimetics. “Modulator” includes inhibitors and activators. Inhibitors are agents that, e.g., bind to, partially or totally block stimulation, decrease, prevent, delay activation, inactivate, desensitize, or down regulate the activity of cyclin dependent kinase, e.g., antagonists. Activators are agents that, e.g., bind to, stimulate, increase, open, activate, facilitate, enhance activation, sensitize or up regulate the activity of cyclin dependent kinase, e.g., agonists. Modulators include agents that, e.g., alter the interaction of cyclin dependent kinase with proteins that bind activators or inhibitors, receptors, including proteins, peptides, lipids, carbohydrates, polysaccharides, or combinations of the above, e.g., lipoproteins, glycoproteins, and the like. Modulators include genetically modified versions of naturally-occurring cyclin dependent kinase protein, e.g., Cdk4, with altered activity, as well as naturally occurring and synthetic ligands, antagonists, agonists, small chemical molecules and the like. Such assays for inhibitors and activators include, e.g., applying putative modulator compounds to a cell expressing cyclin dependent kinase protein, e.g., Cdk4, and then determining the functional effects on viral infection in the cell, as described herein. Samples or assays comprising cyclin dependent kinase protein, e.g., Cdk4, that are treated with a potential activator, inhibitor, or modulator are compared to control samples without the inhibitor, activator, or modulator to examine the extent of inhibition. Control samples (untreated with inhibitors) can be assigned a relative cyclin dependent kinase activity value of 100%. Inhibition of viral infection is achieved when the cyclin dependent kinase activity value relative to the control is about 80%, optionally 50% or 25-0%.

In some particular aspects, examples of small chemical compound inhibitors of Cdks, i.e., Cdk4, is flavopiridol, indolinone, oxindole 91, N⁶-isopentenyladenine, olomoucine, (R)-Roscovitine, hymenialdisine, fascaplysin, compound 66, purvalanol A, purvalanol B, indirubin-3′monoxime, indirubin-5-sulfonate, SU9516, compound 26a, compound 15b, alsterpaullone, quinazoline 51, CINK4, PD0183812, NSC 625987, PD 0332991, aminopurvalanol, Calyculin phosphatase inhibitor. For a review of inhibitors of cyclin dependent kinases, see Knockaert et al., TRENDS in Pharm. Sci. 23: 417-422, 2002; Sridhar et al., The AAPS Journal 8: Article 25, 2005; http://ww.aapsj.org; Shapiro et al., J. Clin. Oncol. 24: 1770-1783, 2006; these references are incorporated by reference in their entireties for all purposes.

“Antagonist” is used in the broadest sense, and includes any molecule that partially or fully blocks, inhibits, or neutralizes a biological activity of cyclin dependent kinase protein, e.g., Cdk4, activity. In a similar manner, the term “agonist” is used in the broadest sense and includes any molecule that mimics or enhances a biological activity of cyclin dependent kinase protein, e.g., Cdk4 activity. Suitable agonist or antagonist molecules specifically include agonist or antagonist antibodies or antibody fragments, fragments or amino acid sequence variants of native cyclin dependent kinase polypeptides, peptides, antisense oligonucleotides, small organic molecules, and the like. Methods for identifying agonists or antagonists of cyclin dependent kinase polypeptides can comprise contacting an cyclin dependent kinase polypeptide with a candidate agonist or antagonist molecule and measuring a detectable change in one or more biological activities normally associated with the cyclin dependent kinase.

“Test compound” refers to a nucleic acid, DNA, RNA, protein, polypeptide, or small chemical entity that is determined to effect an increase or decrease in a gene expression or actin cytoskeleton rearrangement as a result of signaling through cyclin dependent kinase protein, e.g., Cdk4. The test compound can be an antisense RNA, ribozyme, polypeptide, or small molecular chemical entity. The term “test compound” can be any small chemical compound, or a biological entity, such as a protein, sugar, nucleic acid or lipid. Typically, test compounds will be small chemical molecules and polypeptides. A “test compound specific for signaling through cyclin dependent kinase” is determined to be a modulator of cyclin dependent kinase activity.

“Cell-based assays” include cyclin dependent kinase binding assays, for example, radioligand or fluorescent ligand binding assays for cyclin dependent kinase activity or binding of the protein, e.g., Cdk4 protein, in cells, plasma membranes, detergent-solubilized plasma membrane proteins, immobilized collagen.

In one aspect, cyclin dependent kinase protein, e.g., Cdk4, can be assayed by either immobilizing the ligand/interacting protein or the kinase. For example, the assay can include immobilizing cyclin dependent kinase fused to a His tag onto Ni-activated NTA resin beads. Inhibitors of cyclin dependent kinase can be added in an appropriate buffer and the beads incubated for a period of time at a given temperature. After washes to remove unbound material, the bound protein can be released with, for example, SDS, buffers with a high pH, and the like and analyzed.

“Contacting” refers to mixing a test compound in a soluble form into an assay system, for example, a cell-based assay system, such that an effect upon receptor-mediated signaling can be measured.

“Signaling in cells” refers to the interaction of a ligand with a kinase, such as cyclin dependent kinase protein, e.g., Cdk4, to produce a response, for example, to prevent or alleviate an inflammatory-related disease or disorder. “Signaling responsiveness” or “effective to activate signaling” or “stimulating a cell-based assay system” refers to the ability of inhibitors of cyclin dependent kinase activity to stimulate an immune response, and to prevent or alleviate an inflammatory-related disease or disorder.

“Detecting an effect” refers to an effect measured in a cell-based assay system. For example, the effect detected can be cyclin dependent kinase protein activity, e.g., Cdk4, in an assay system, for example, a Jurkat cell in vitro assay or a human CD4⁺ T cell in vitro assay.

“Assay being indicative of modulation” refers to results of a cell-based assay system indicating that cell activation by cyclin dependent kinase protein, e.g., Cdk4, induces a protective response in cells against an inflammatory-related disease or disorder.

“Biological activity” and “biologically active” with regard to an inhibitor of cyclin dependent kinase protein, e.g., Cdk4, of the present invention refer to the ability of the inhibitor molecule to specifically bind to and signal through a native or recombinant cyclin dependent kinase, or to block the ability of a native or recombinant cyclin dependent kinase to participate in signal transduction. As discussed above, Cdk4 plays a major role in regulating cell cycle proliferation, and gene expression through phosphorylation of Retinoblastoma (Rb) protein (biological acitivities associated with Cdk4). In addition, Cdk4 also regulates cell death, differentiation, and leukocyte adhesion regulation. Thus, the (native and variant) ligands of cyclin dependent kinase of the present invention include agonists and antagonists of a native or recombinant cyclin dependent kinase. Preferred biological activities of the ligands of cyclin dependent kinase protein, e.g., Cdk4, of the present invention include the ability to enhance an immune response, or treat an inflammatory-related disease or disorder. Accordingly, the administration of the compounds or agents of the present invention can prevent or delay, to alleviate, or to arrest or inhibit development of the symptoms or conditions associated with an inflammatory-related disease or disorder, or other disorders.

“Subpopulations of T lymphocytes” or “T cell subset(s)” refers to T lymphocytes or T cells characterized by the expression of particular cell surface markers (see Barclay et al., (eds.), THE LEUKOCYTE ANTIGEN FACTS BOOK, 2^{nd} ed., Academic Press, London, United Kingdom, 1997; this reference is herein incorporated by reference for all purposes).

“Epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

An intact “antibody” comprises at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, C_{H}1, C_{H}2 and C_{H}3. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) through cellular receptors such as Fc receptors (e.g., FcγRI, FcγRIIa, FcγRIIb, FcγRIII, and FcRη) and the first component (Clq) of the classical complement system. The term antibody includes antigen-binding portions of an intact antibody that retain capacity to bind the antigen. Examples of antigen binding portions include (i) a Fab fragment, a monovalent fragment consisting of the V_{L}, V_{H}, CL and C_{H}1 domains; (ii) a F(ab′)₂ fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al., Nature 341: 544-546, 1989), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); See, e.g., Bird et al., Science 242: 423-426, 1988; Huston et al., Proc. Natl. Acad. Sci. U.S.A. 85: 5879-5883, 1988). Such single chain antibodies are included by reference to the term “antibody”. Fragments can be prepared by recombinant techniques or enzymatic or chemical cleavage of intact antibodies.

“Human sequence antibody” includes antibodies having variable and constant regions (if present) derived from human immunoglobulin sequences. The human sequence antibodies of the invention can include amino acid residues not encoded by human germline immunoglobulin sequences (e.g., mutations introduced by random or site-specific mutagenesis in vitro or by somatic mutation in vivo). However, the term “human sequence antibody”, as used herein, is not intended to include antibodies in which entire CDR sequences sufficient to confer antigen specificity and derived from the germline of another mammalian species, such as a mouse, have been grafted onto human framework sequences (i.e., humanized antibodies).

“Monoclonal antibody” or “monoclonal antibody composition” refer to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions (if present) derived from human germline immunoglobulin sequences. In one aspect, the human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

“Diclonal antibody” refers to a preparation of at least two antibodies to an antigen. Typically, the different antibodies bind different epitopes.

“Oligoclonal antibody” refers to a preparation of 3 to 100 different antibodies to an antigen. Typically, the antibodies in such a preparation bind to a range of different epitopes.

“Polyclonal antibody” refers to a preparation of more than 1 (two or more) different antibodies to an antigen. Such a preparation includes antibodies binding to a range of different epitopes.

“Recombinant human antibody” includes all human sequence antibodies of the invention that are prepared, expressed, created or isolated by recombinant means, such as antibodies isolated from an animal (e.g., a mouse) that is transgenic for human immunoglobulin genes (described further below); antibodies expressed using a recombinant expression vector transfected into a host cell, antibodies isolated from a recombinant, combinatorial human antibody library, or antibodies prepared, expressed, created or isolated by any other means that involves splicing of human immunoglobulin gene sequences to other DNA sequences. Such recombinant human antibodies have variable and constant regions (if present) derived from human germline immunoglobulin sequences. Such antibodies can, however, be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

A “eterologous antibody” is defined in relation to the transgenic non-human organism producing such an antibody. This term refers to an antibody having an amino acid sequence or an encoding nucleic acid sequence corresponding to that found in an organism not consisting of the transgenic non-human animal, and generally from a species other than that of the transgenic non-human animal.

A “heterohybrid antibody” refers to an antibody having a light and heavy chains of different organismal origins. For example, an antibody having a human heavy chain associated with a murine light chain is a heterohybrid antibody.

“Substantially pure” or “isolated” means an object species (e.g., an antibody of the invention) has been identified and separated and/or recovered from a component of its natural environment such that the object species is the predominant species present (i.e., on a molar basis it is more abundant than any other individual species in the composition); a “substantially pure” or “isolated” composition also means where the object species comprises at least about 50 percent (on a molar basis) of all macromolecular species present. A substantially pure or isolated composition can also comprise more than about 80 to 90 percent by weight of all macromolecular species present in the composition. An isolated object species (e.g., antibodies of the invention) can also be purified to essential homogeneity (contaminant species cannot be detected in the composition by conventional detection methods) wherein the composition consists essentially of derivatives of a single macromolecular species. For example, an isolated antibody to cyclin dependent kinase can be substantially free of other antibodies that lack binding to human cyclin dependent kinase and bind to a different antigen. Further, an isolated antibody that specifically binds to an epitope, isoform or variant of human cyclin dependent kinase may, however, have cross-reactivity to other related antigens, e.g., from other species (e.g., cyclin dependent kinase species homologs). Moreover, an isolated antibody of the invention be substantially free of other cellular material (e.g., non-immunoglobulin associated proteins) and/or chemicals.

“Specific binding” refers to preferential binding of an antibody to a specified antigen relative to other non-specified antigens. The phrase “specifically (or selectively) binds” to an antibody refers to a binding reaction that is determinative of the presence of the protein in a heterogeneous population of proteins and other biologics. Typically, the antibody binds with an association constant (Kₐ) of at least about 1×10⁶ M⁻¹ or 10⁷ M⁻¹, or about 10⁸ M⁻¹ to 10⁹ M⁻¹, or about 10¹⁰ M⁻¹ to 10¹¹ M⁻¹ or higher, and binds to the specified antigen with an affinity that is at least two-fold greater than its affinity for binding to a non-specific antigen (e.g., BSA, casein) other than the specified antigen or a closely-related antigen. The phrases “an antibody recognizing an antigen” and “an antibody specific for an antigen” are used interchangeably herein with the term “an antibody which binds specifically to an antigen”. A predetermined antigen is an antigen that is chosen prior to the selection of an antibody that binds to that antigen.

“Specifically bind(s)” or “bind(s) specifically” when referring to a peptide refers to a peptide molecule which has intermediate or high binding affinity, exclusively or predominately, to a target molecule. The phrases “specifically binds to” refers to a binding reaction which is determinative of the presence of a target protein in the presence of a heterogeneous population of proteins and other biologics. Thus, under designated assay conditions, the specified binding moieties bind preferentially to a particular target protein and do not bind in a significant amount to other components present in a test sample. Specific binding to a target protein under such conditions can require a binding moiety that is selected for its specificity for a particular target antigen. A variety of assay formats can be used to select ligands that are specifically reactive with a particular protein. For example, solid-phase ELISA immunoassays, immunoprecipitation, Biacorem and Western blot are used to identify peptides that specifically react with the antigen. Typically a specific or selective reaction will be at least twice background signal or noise and more typically more than 10 times background.

“High affinity” for an antibody refers to an equilibrium association constant (Kₐ) of at least about 10⁷M⁻¹, at least about 10⁸M⁻¹, at least about 10⁹M⁻¹, at least about 10¹⁰M⁻¹, at least about 10¹¹M⁻¹, or at least about 10¹²M⁻¹ or greater, e.g., up to 10¹³M⁻¹ or 10¹⁴M⁻¹ or greater. However, “high affinity” binding can vary for other antibody isotypes.

The term “Kₐ”, as used herein, is intended to refer to the equilibrium association constant of a particular antibody-antigen interaction. This constant has units of 1/M.

The term “K_{d}”, as used herein, is intended to refer to the equilibrium dissociation constant of a particular antibody-antigen interaction. This constant has units of M.

The term “kₐ”, as used herein, is intended to refer to the kinetic association constant of a particular antibody-antigen interaction. This constant has units of 1/Ms.

The term “k_{d}”, as used herein, is intended to refer to the kinetic dissociation constant of a particular antibody-antigen interaction. This constant has units of 1/s.

“Particular antibody-antigen interactions” refers to the experimental conditions under which the equilibrium and kinetic constants are measured.

“Isotype” refers to the antibody class that is encoded by heavy chain constant region genes. Heavy chains are classified as gamma, mu, alpha, delta, or epsilon, and define the antibody's isotype as IgG, IgM, IgA, IgD and IgE, respectively. Additional structural variations characterize distinct subtypes of IgG (e.g., IgG₁, IgG₂, IgG₃ and IgG₄) and IgA (e.g., IgA₁ and IgA₂)

“Isotype switching” refers to the phenomenon by which the class, or isotype, of an antibody changes from one Ig class to one of the other Ig classes.

“Nonswitched isotype” refers to the isotypic class of heavy chain that is produced when no isotype switching has taken place; the C_{H} gene encoding the nonswitched isotype is typically the first C_{H} gene immediately downstream from the functionally rearranged VDJ gene. Isotype switching has been classified as classical or non-classical isotype switching. Classical isotype switching occurs by recombination events which involve at least one switch sequence region in the transgene. Non-classical isotype switching can occur by, for example, homologous recombination between human σ_{μ} and human Σ_{μ} (δ-associated deletion). Alternative non-classical switching mechanisms, such as intertransgene and/or interchromosomal recombination, among others, can occur and effectuate isotype switching.

“Switch sequence” refers to those DNA sequences responsible for switch recombination. A “switch donor” sequence, typically a μ switch region, are 5′ (i.e., upstream) of the construct region to be deleted during the switch recombination. The “switch acceptor” region are between the construct region to be deleted and the replacement constant region (e.g., γ, ε, and alike). As there is no specific site where recombination always occurs, the final gene sequence is not typically predictable from the construct.

“Glycosylation pattern” is defined as the pattern of carbohydrate units that are covalently attached to a protein, more specifically to an immunoglobulin protein. A glycosylation pattern of a heterologous antibody can be characterized as being substantially similar to glycosylation patterns which occur naturally on antibodies produced by the species of the non-human transgenic animal, when one of ordinary skill in the art would recognize the glycosylation pattern of the heterologous antibody as being more similar to said pattern of glycosylation in the species of the non-human transgenic animal than to the species from which the C_{H} genes of the transgene were derived.

“Naturally-occurring” as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

“Immunoglobulin locus” refers to a genetic element or set of linked genetic elements that comprise information that can be used by a B cell or B cell precursor to express an immunoglobulin peptide. This peptide can be a heavy chain peptide, a light chain peptide, or the fusion of a heavy and a light chain peptide. In the case of an unrearranged locus, the genetic elements are assembled by a B cell precursor to form the gene encoding an immunoglobulin peptide. In the case of a rearranged locus, a gene encoding an immunoglobulin peptide is contained within the locus.

“Rearranged” refers to a configuration of a heavy chain or light chain immunoglobulin locus wherein a V segment is positioned immediately adjacent to a D-J or J segment in a conformation encoding essentially a complete V_{H} or V_{L} domain, respectively. A rearranged immunoglobulin gene locus can be identified by comparison to germline DNA; a rearranged locus has at least one recombined heptamer/nonamer homology element.

“Unrearranged” or “germline configuration” in reference to a V segment refers to the configuration wherein the V segment is not recombined so as to be immediately adjacent to a D or J segment.

“Nucleic acid” or “nucleic acid molecule” refer to a deoxyribonucleotide or ribonucleotide polymer in either single- or double-stranded form, and unless otherwise limited, can encompass known analogs of natural nucleotides that can function in a similar manner as naturally occurring nucleotides.

“Isolated nucleic acid” in reference to nucleic acids encoding antibodies or antibody portions (e.g., V_{H}, V_{L}, CDR3) that bind to the antigen, is intended to refer to a nucleic acid in which the nucleotide sequences encoding the antibody or antibody portion are free of other nucleotide sequences encoding antibodies or antibody portions that bind antigens other than, for example, cyclin dependent kinase, which other sequences can naturally flank the nucleic acid in human genomic DNA.

“Substantially identical,” in the context of two nucleic acids or polypeptides refers to two or more sequences or subsequences that have at least about 80%, about 90%, about 95% or higher nucleotide or amino acid residue identity, when compared and aligned for maximum correspondence, as measured using the following sequence comparison method and/or by visual inspection. Such “substantially identical” sequences are typically considered to be homologous. The “substantial identity” can exist over a region of sequence that is at least about 50 residues in length, over a region of at least about 100 residues, or over a region at least about 150 residues, or over the full length of the two sequences to be compared. As described below, any two antibody sequences can only be aligned in one way, by using the numbering scheme in Kabat. Therefore, for antibodies, percent identity has a unique and well-defined meaning.

Amino acids from the variable regions of the mature heavy and light chains of immunoglobulins are designated Hx and Lx respectively, where x is a number designating the position of an amino acid according to the scheme of Kabat, Sequences of Proteins of Immunological Interest (National Institutes of Health, Bethesda, Md., 1987 and 1991). Kabat lists many amino acid sequences for antibodies for each subgroup, and lists the most commonly occurring amino acid for each residue position in that subgroup to generate a consensus sequence. Kabat uses a method for assigning a residue number to each amino acid in a listed sequence, and this method for assigning residue numbers has become standard in the field. Kabat's scheme is extendible to other antibodies not included in his compendium by aligning the antibody in question with one of the consensus sequences in Kabat by reference to conserved amino acids. The use of the Kabat numbering system readily identifies amino acids at equivalent positions in different antibodies. For example, an amino acid at the L50 position of a human antibody occupies the equivalent position to an amino acid position L50 of a mouse antibody. Likewise, nucleic acids encoding antibody chains are aligned when the amino acid sequences encoded by the respective nucleic acids are aligned according to the Kabat numbering convention. An alternative structural definition has been proposed by Chothia et al., J. Mol. Biol 196: 901-917, 1987; Chothia et al., Nature 342: 878-883, 1989; and Chothia et al., J. Mol. Biol 186: 651-663, 1989, which are herein incorporated by reference for all purposes.

The nucleic acids of the invention be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art (See, e.g., Sambrook et al., MOLECULAR CLONING, A LABORATORY MANUAL, 2^{nd} ed., 1989; Tijssen (1993); and Ausubel (1994), incorporated by reference for all purposes). The nucleic acid sequences of the invention and other nucleic acids used to practice this invention, whether RNA, cDNA, genomic DNA, or hybrids thereof, can be isolated from a variety of sources, genetically engineered, amplified, and/or expressed recombinantly. Any recombinant expression system can be used, including, in addition to bacterial, e.g., yeast, insect or mammalian systems. Alternatively, these nucleic acids can be chemically synthesized in vitro. Techniques for the manipulation of nucleic acids, such as, e.g., subcloning into expression vectors, labeling probes, sequencing, and hybridization are well described in the scientific and patent literature, see, e.g., Sambrook et al., 1989. Nucleic acids can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, such as fluid or gel precipitin reactions, immunodiffusion (single or double), immunoelectrophoresis, radioimmunoassay (RIAs), enzyme-linked immunosorbent assays (ELISAs), immuno-fluorescent assays, Southern analysis, Northern analysis, dot-blot analysis, gel electrophoresis (e.g., SDS-PAGE), RT-PCR, quantitative PCR, other nucleic acid or target or signal amplification methods, radiolabeling, scintillation counting, and affinity chromatography.

The nucleic acid compositions of the present invention, while often in a native sequence (except for modified restriction sites and the like), from either cDNA, genomic or mixtures can be mutated, thereof in accordance with standard techniques to provide gene sequences. For coding sequences, these mutations, can affect amino acid sequence as desired. In particular, DNA sequences substantially homologous to or derived from native V, D, J, constant, switches and other such sequences described herein are contemplated (where “derived” indicates that a sequence is identical or modified from another sequence).

“Recombinant host cell” or “host cell” refers to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

A “label” is a composition detectable by spectroscopic, photochemical, biochemical, immunochemical, or chemical means. For example, useful labels include ³²P, fluorescent dyes, electron-dense reagents, enzymes (e.g., as commonly used in an ELISA), biotin, digoxigenin, or haptens and proteins for which antisera or monoclonal antibodies are available (e.g., the polypeptides of the invention can be made detectable, e.g., by incorporating a radiolabel into the peptide, and used to detect antibodies specifically reactive with the peptide).

“Sorting” in the context of cells as used herein to refers to both physical sorting of the cells, as can be accomplished using, e.g., a fluorescence activated cell sorter, as well as to analysis of cells based on expression of cell surface markers, e.g., FACS analysis in the absence of sorting.

Components of an immune response can be detected in vitro by various methods that are well known to those of ordinary skill in the art. For example, (1) cytotoxic T lymphocytes can be incubated with radioactively labeled target cells and the lysis of these target cells detected by the release of radioactivity, (2) helper T lymphocytes can be incubated with antigens and antigen presenting cells and the synthesis and secretion of cytokines and proliferation as measured by assays described below and measured by standard methods (Windhagen et al., Immunity 2: 373-380, 1995), (3) antigen presenting cells can be incubated with whole protein antigen and the presentation of that antigen on MHC detected by either T lymphocyte activation assays or biophysical methods (Harding et al., Proc. Natl. Acad. Sci. U.S.A., 86: 4230-4, 1989), (4) mast cells can be incubated with reagents that cross-link their Fc-epsilon receptors and histamine release measured by enzyme immunoassay. Siraganian et al., TIPS 4: 432-437, 1983.

Similarly, products of an immune response in either a model organism (e.g., mouse) or a human patient can also be detected by various methods that are well known to those of ordinary skill in the art. For example, (1) the production of antibodies in response to vaccination can be readily detected by standard methods currently used in clinical laboratories, e.g., an ELISA; (2) the migration of immune cells to sites of inflammation can be detected by scratching the surface of skin and placing a sterile container to capture the migrating cells over scratch site (Peters et al., Blood 72:1310-5, 1988); (3) the proliferation of peripheral blood mononuclear cells in response to mitogens or mixed lymphocyte reaction can be measured using ³H-thymidine; (4) the phagocitic capacity of granulocytes, macrophages, and other phagocytes in PBMCs can be measured by placing PMBCs in wells together with labeled particles (Peters et al., 1988); and (5) the radioimmunoassay of immune system cells can be measured by labeling PBMCs with antibodies to CD molecules such as CD4 and CD8 and measuring the fraction of the PBMCs expressing these markers.

“Signal transduction pathway” or “signal transduction event” refers to at least one biochemical reaction, but more commonly a series of biochemical reactions, which result from interaction of a cell with a stimulatory compound or agent. Thus, the interaction of a stimulatory compound with a cell generates a “signal” that is transmitted through the signal transduction pathway, ultimately resulting in a cellular response, e.g., an immune response described above.

A signal transduction pathway refers to the biochemical relationship between a variety of signal transduction molecules that play a role in the transmission of a signal from one portion of a cell to another portion of a cell. As used herein, the phrase “cell surface receptor” includes molecules and complexes of molecules capable of receiving a signal and the transmission of such a signal across the plasma membrane of a cell. An example of a “cell surface receptor” is the T cell receptor (TCR) or the B7 ligands of CTLA-4/CD28.

A signal transduction pathway in a cell can be initiated by interaction of a cell with a stimulator that is inside or outside of the cell. If an exterior (i.e., outside of the cell) stimulator (e.g., an MHC-antigen complex on an antigen presenting cell) interacts with a cell surface receptor (e.g., a T cell receptor), a signal transduction pathway can transmit a signal across the cell's membrane, through the cytoplasm of the cell, and in some instances into the nucleus. If an interior (e.g., inside the cell) stimulator interacts with an intracellular signal transduction molecule, a signal transduction pathway can result in transmission of a signal through the cell's cytoplasm, and in some instances into the cell's nucleus.

Signal transduction can occur through, e.g., the phosphorylation of a molecule; non-covalent allosteric interactions; complexing of molecules; change of protein localization; the conformational change of a molecule; calcium release; inositol phosphate production; proteolytic cleavage; cyclic nucleotide production and diacylglyceride production. Typically, signal transduction occurs through phosphorylating a signal transduction molecule.

“Nonspecific T cell activation” refers to the stimulation of T cells independent of their antigenic specificity.

This invention relies on routine techniques in the field of recombinant genetics. Basic texts disclosing the general methods of use in this invention include Sambrook et al., MOLECULAR CLONING, A LABORATORY MANUAL, 2^{nd} ed., 1989; Kriegler, GENE TRANSFER AND EXPRESSION: A LABORATORY MANUAL, 1990; and Ausubel et al., eds., CURRENT PROTOCOLS IN MOLECULAR BIOLOGY, 1994.

Cyclin dependent kinase, e.g., Cdk4, nucleic acids, polymorphic variants, orthologs, and alleles that are substantially identical to sequences provided herein can be isolated using nucleic acid probes and oligonucleotides of cyclin dependent kinase, e.g., Cdk4, under stringent hybridization conditions, by screening libraries. Alternatively, expression libraries can be used to isolate cyclin dependent kinase protein, or protein encoding cyclin dependent kinase polymorphic variants, orthologs, and alleles by detecting expressed homologs immunologically with antisera or purified antibodies made against human cyclin dependent kinase, or portions thereof.

### Peptides and Polypeptides

The invention provides isolated or recombinant polypeptides comprising an amino acid sequence having at least 95%, 96%, 97%, 98%, 99% or more sequence identity to Cdk4 over a region of at least about 100, 150, 200, 250, 300, 350, 400, 450, 500, 550, 600, 650, 700, 750, 800, 850, 900, 950, 1000, 1100 or more residues, or, the full length of the polypeptide, or, a polypeptide encoded by a nucleic acid of the invention. The invention provides methods for inhibiting the activity of cyclin dependent kinase polypeptide, e.g., Cdk4 polypeptides, e.g., a polypeptide of the invention. The invention also provides methods for screening for compositions that inhibit the activity of, or bind to (e.g., bind to the active site), of cyclin dependent kinase polypeptides, e.g., a polypeptide of the invention.

In one aspect, the invention provides cyclin dependent kinase polypeptides (and the nucleic acids encoding them) where one, some or all of the cyclin dependent kinase polypeptides replacement with substituted amino acids. In one aspect, the invention provides methods to disrupt the interaction of cyclin dependent kinase polypeptides with other proteins, in pathways related to entry or replication of infectious viruses in the cells.

The peptides and polypeptides of the invention can be expressed recombinantly in vivo after administration of nucleic acids, as described above, or, they can be administered directly, e.g., as a pharmaceutical composition. They can be expressed in vitro or in vivo to screen for modulators of a cyclin dependent kinase activity and for agents that can treat or ameliorate an inflammatory-related disease or disorder.

Polypeptides and peptides of the invention can be isolated from natural sources, be synthetic, or be recombinantly generated polypeptides. Peptides and proteins can be recombinantly expressed in vitro or in vivo. The peptides and polypeptides of the invention can be made and isolated using any method known in the art. Polypeptide and peptides of the invention can also be synthesized, whole or in part, using chemical methods well known in the art (see e.g., Caruthers, Nucleic Acids Res. Symp. Ser. 215-223, 1980; Horn, Nucleic Acids Res. Symp. Ser. 225-232, 1980; Banga, THERAPEUTIC PEPTIDES AND PROTEINS, FORMULATION, PROCESSING AND DELIVERY SYSTEMS TECHNOMIC PUBLISHING CO., Lancaster, Pa., 1995). For example, peptide synthesis can be performed using various solid-phase techniques (see e.g., Roberge, Science 269: 202, 1995; Merrifield, Methods Enzymol. 289: 3-13, 1997) and automated synthesis can be achieved, e.g., using the ABI 431A Peptide Synthesizer (Perkin Elmer) in accordance with the instructions provided by the manufacturer.

The peptides and polypeptides of the invention, as defined above, include all “mimetic” and “peptidomimetic” forms. The terms “mimetic” and “peptidomimetic” refer to a synthetic chemical compound which has substantially the same structural and/or functional characteristics of the polypeptides of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. As with polypeptides of the invention which are conservative variants, routine experimentation will determine whether a mimetic is within the scope of the invention, i.e., that its structure and/or function is not substantially altered. Thus, a mimetic composition is within the scope of the invention if, when administered to or expressed in a cell, it has a cyclin dependent kinase activity.

In one aspect, the polypeptide or peptidomimetic composition can be a dominant-negative mutant within the scope of the invention if it can inhibit an activity of a cyclin dependent kinase polypeptide, e.g., Cdk4 polypeptides of the invention, e.g., be a dominant-negative mutant or bind to an antibody of the invention. The dominant negative mutant can be a peptide or peptide mimetic that can inhibit an activity of a cyclin dependent kinase, or a nucleic acid composition, in the form of a DNA vector or gene therapy vector, that expresses a dominant-negative polypeptide that can inhibit an activity of a cyclin dependent kinase. The dominant negative mutant can bind to a ligand of the kinase or a target target, affecting ligand target interaction. The dominant negative molecule can act, for example, by blocking protein protein interactions, or by blocking phosphorylation of the kinase. An example of a dominant negative peptide is a peptide with a mutation in a lysine residue in the ATP binding domain of the cyclin dependent kinase, as described herein, that inhibits cyclin dependent kinase activity. A further example of a dominant negative peptide is a peptide with a mutation in the SH2 domain or SH3 domain of the cyclin dependent kinase as described herein, that inhibits cyclin dependent kinase activity.

Polypeptide mimetic compositions can contain any combination of non-natural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. For example, a polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in CHEMISTRY AND BIOCHEMISTRY OF AMiNO ACIDS, PEPTIDES AND PROTEINS, Vol. 7, pp 267-357, “Peptide Backbone Modifications,” Marcell Dekker, NY).

A polypeptide can also be characterized as a mimetic by containing all or some non-natural residues in place of naturally occurring amino acid residues. Non-natural residues are well described in the scientific and patent literature; a few exemplary non-natural compositions useful as mimetics of natural amino acid residues and guidelines are described below. Mimetics of aromatic amino acids can be generated by replacing by, e.g., D- or L-naphylalanine; D- or L-phenylglycine; D- or L-2 thieneylalanine; D- or L-1, -2,3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; K- or L-p-methoxy-biphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings.

Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R′—N—C—N—R′) such as, e.g., 1-cyclohexyl-3(2-morpholin-yl-(4-ethyl)carbodiimide or 1-ethyl-3(4-azonia-4,4-dimetholpentyl)carbodiimide. Aspartyl or glutamyl can also be converted to asparaginyl and glutaminyl residues by reaction with ammonium ions.

Mimetics of basic amino acids can be generated by substitution with, e.g., (in addition to lysine and arginine) the amino acids ornithine, citrulline, or (guanidino)-acetic acid, or (guanidino)alkyl-acetic acid, where alkyl is defined above. Nitrile derivative (e.g., containing the CN-moiety in place of COOH) can be substituted for aspargine or glutamine. Asparaginyl and glutaminyl residues can be deaminated to the corresponding aspartyl or glutamyl residues.

Arginine residue mimetics can be generated by reacting arginyl with, e.g., one or more conventional reagents, including, e.g., phenylglyoxal, 2,3-butanedione, 1,2-cyclohexanedione, or ninhydrin, preferably under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl)propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitrobenzenesulfonic acid, O-methylisourea, 2,4, pentanedione, and transamidase-catalyzed reactions with glyoxylate. Mimetics of methionine can be generated by reaction with, e.g., methionine sulfoxide. Mimetics of □adioim include, e.g., pipecolic acid, thiazolidine carboxylic acid, 3- or 4-hydroxy guanidino, dehydroproline, 3- or 4-methylproline, or 3,3,-dimethylproline. Histidine residue mimetics can be generated by reacting histidyl with, e.g., diethylprocarbonate or para-bromophenacyl bromide. Other mimetics include, e.g., those generated by hydroxylation of guanidino and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

A component of a polypeptide of the invention can also be replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, referred to as the D-amino acid, but which can additionally be referred to as the R- or S-form

The invention also provides polypeptides that are “substantially identical” to an exemplary polypeptide of the invention. A “substantially identical” amino acid sequence is a sequence that differs from a reference sequence by one or more conservative or non-conservative amino acid substitutions, deletions, or insertions, particularly when such a substitution occurs at a site that is not the active site of the molecule, and provided that the polypeptide essentially retains its functional properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for asparagine). One or more amino acids can be deleted, for example, from a cyclin dependent kinase polypeptide of the invention, resulting in modification of the structure of the polypeptide, without significantly altering its biological activity. For example, amino- or carboxyl-terminal, or internal, amino acids which are not required for a cyclin dependent kinase activity or interaction can be removed.

The skilled artisan will recognize that individual synthetic residues and polypeptides incorporating these mimetics can be synthesized using a variety of procedures and methodologies, which are well described in the scientific and patent literature, e.g., Organic Syntheses Collective Volumes, Gilman et al. (Eds) John Wiley & Sons, Inc., NY. Peptides and peptide mimetics of the invention can also be synthesized using combinatorial methodologies. Various techniques for generation of peptide and peptidomimetic libraries are well known, and include, e.g., multipin, tea bag, and split-couple-mix techniques; (see, e.g., al-Obeidi, Mol. Biotechnol. 9: 205-223, 1998; Hruby, Curr. Opin. Chem. Biol. 1: 114-119, 1997; Ostergaard, Mol. Divers. 3: 17-27, 1997; Ostresh, Methods Enzymol. 267: 220-234, 1996). Modified peptides of the invention can be further produced by chemical modification methods, (see, e.g., Belousov, Nucleic Acids Res 25: 3440-3444, 1997; Frenkel, Free Radic. Biol. Med 19: 373-380, 1995; Blommers, Biochemistry 33: 7886-7896, 1994).

Peptides and polypeptides of the invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Amgen Inc., Seattle Wash.). The inclusion of a cleavable linker sequences such as Factor Xa or enterokinase (Invitrogen, San Diego Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see e.g., Williams, Biochemistry 34: 1787-1797, 1995; Dobeli, Protein Expr. Purif. 12: 404-14, 1998). The histidine residues facilitate detection and purification while the enterokinase cleavage site provides a means for purifying the epitope from the remainder of the fusion protein. Technology pertaining to vectors encoding fusion proteins and application of fusion proteins are well described in the scientific and patent literature (see e.g., Kroll, DNA Cell. Biol. 12: 441-53, 1993).

Some peptides have an ability to cross the cell membrane and enter a cell. These peptides, termed “protein transduction domains” (PTDs), can be linked to a cargo moiety and can transport the cargo moiety across the cell membrane and into the cell. Such transport is termed “peptide transport” because the peptides transport cargo moieties across the cell membrane and into the cell. Cargo moieties of the invention can be small molecules, proteins, or nucleic acids. One well characterized protein domain (PTD) is a tat derived peptide (Frankel et al., U.S. Pat. Nos. 5,804,604, 5,747,641, 5,674,980, 5,670,617, and 5,652,122). Transducing peptides derived from Antennapedia (Antp), TAT-HIV, and VP22 can penetrate biological membranes, act as cargo vehicles, and target to specific subcellular compartments. PTDs are effective in transporting small molecules, proteins, and nucleic acids of the invention, for example, into leukocytes.

“Polypeptide” and “protein” as used herein, refer to amino acids joined to each other by peptide bonds or modified peptide bonds, i.e., peptide isosteres, and can contain modified amino acids other than the 20 gene-encoded amino acids. The term “polypeptide” also includes peptides and polypeptide fragments, motifs and the like. The term also includes glycosylated polypeptides. The peptides and polypeptides of the invention also include all “mimetic” and “peptidomimetic” forms, as described in further detail, below.

“Isolated” means that the material is removed from its original environment (e.g., the natural environment if it is naturally occurring). For example, a naturally occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment. As used herein, an isolated material or composition can also be a “purified” composition, i.e., it does not require absolute purity; rather, it is intended as a relative definition. Individual nucleic acids obtained from a library can be conventionally purified to electrophoretic homogeneity. In alternative aspects, the invention provides nucleic acids which have been purified from genomic DNA or from other sequences in a library or other environment by at least one, two, three, four, five or more orders of magnitude.

### Labels

The particular label or detectable group used in the assay is not a critical aspect of the invention, so long as it does not significantly interfere with the specific binding of the small molecule chemical inhibitors or siRNA inhibitors of cyclin dependent kinase activity, e.g., Cdk4 activity, ligand mimetics, derivatives and analogs thereof, antibodies, or nucleic acid compositions, e.g., antisense oligonucleotides or double stranded RNA oligonucleotides (RNAi), used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g. Dynabeads™), fluorescent dyes (e.g., fluorescein isothiocyanate, Texas red, rhodamine, and the like), radiolabels (e.g., ³H, ¹⁴C, ³⁵S, ¹²⁵I, ¹²¹, ¹¹²In, ⁹⁹mTc), other imaging agents such as microbubbles (for ultrasound imaging), ¹⁸F, ¹¹C, ¹⁵O, (for Positron emission tomography), ^{99m}TC, ¹¹¹In (for Single photon emission tomography), enzymes (e.g., horse radish peroxidase, alkaline phosphatase and others commonly used in an ELISA), and calorimetric labels such as colloidal gold or colored glass or plastic (e.g. polystyrene, polypropylene, latex, and the like) beads. Patents that described the use of such labels include U.S. Pat. Nos. 3,817,837; 3,850,752; 3,939,350; 3,996,345; 4,277,437; 4,275,149; and 4,366,241, each incorporated herein by reference in their entirety and for all purposes (see also HANDBOOK OF FLUORESCENT PROBES AND RESEARCH CHEMICALS (6^{th} Ed., Molecular Probes, Inc., Eugene Oreg.)).

The label may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands can be used. Where a ligand has a natural anti-ligand, for example, biotin, thyroxine, and cortisol, it can be used in conjunction with the labeled, naturally occurring anti-ligands. Alternatively, any haptenic or antigenic compound can be used in combination with an antibody.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidoreductases, particularly peroxidases. Fluorescent compounds include fluorescein and its derivatives, rhodamine and its derivatives, dansyl, umbelliferone, and the like Chemiluminescent compounds include luciferin, and 2,3-dihydrophthalazinediones, e.g., luminol. For a review of various labeling or signal producing systems which may be used, see, U.S. Pat. No. 4,391,904, incorporated herein by reference in its entirety and for all purposes.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple calorimetric labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

Frequently, the cyclin dependent kinase polypeptide, e.g., Cdk4 polypeptides will be labeled by joining, either covalently or non-covalently, a substance which provides for a detectable signal.

### Therapeutic Applications

The small chemical molecule, siRNA molecule, dominant-negative mutants, or antibody inhibitors of cyclin dependent kinase identified by the methods of the present invention can be used in a variety of methods of treatment. Thus, the present invention provides compositions and methods for treating inflammatory-related diseases or disorders.

### Pharmaceutical Compositions

Small molecule chemical inhibitors, siRNA inhibitors, or dominant negative mutants of cyclin dependent kinase activity, e.g., Cdk4 activity, ligand mimetics, derivatives and analogs thereof, antibodies, or nucleic acid compositions, e.g., antisense oligonucleotides or double stranded RNA oligonucleotides (RNAi), useful in the present compositions and methods can be administered to a human patient per se, in the form of a stereoisomer, prodrug, pharmaceutically acceptable salt, hydrate, solvate, acid salt hydrate, N-oxide or isomorphic crystalline form thereof, or in the form of a pharmaceutical composition where the compound is mixed with suitable carriers or excipient(s) in a therapeutically effective amount, for example, cancer or metastatic cancer.

Pharmaceutically acceptable carriers are determined in part by the particular composition being administered, as well as by the particular method used to administer the composition. Accordingly, there is a wide variety of suitable formulations of pharmaceutical compositions for administering the antibody compositions (see, e.g., REMINGTON: THE SCIENCE AND PRACTICE OF PHARMACY, 20^{th} Ed., Gennaro, A. R. (ed.), Mack Publishing Company, Easton, Pa., 2000, incorporated herein by reference). The pharmaceutical compositions generally comprise a differentially expressed protein, agonist or antagonist in a form suitable for administration to a patient. The pharmaceutical compositions are generally formulated as sterile, substantially isotonic and in full compliance with all Good Manufacturing Practice (GMP) regulations of the U.S. Food and Drug Administration.

A wide variety of compositions and administration methods may be used in conjunction with the modulators of the present invention. Such compositions can include, in addition to the modulators of the present invention, conventional pharmaceutical excipients, and other conventional, pharmaceutically inactive agents. Additionally, the compositions may include active agents in addition to the modulators of the present invention. These additional active agents may include additional compounds according to the invention, and/or one or more other pharmaceutically active agents.

The compositions may be in gaseous, liquid, semi-liquid or solid form, formulated in a manner suitable for the route of administration to be used. For oral administration, capsules and tablets are typically used. For parenteral administration, reconstitution of a lyophilized powder, prepared as described herein, is typically used.

Compositions comprising modulators of the present invention may be administered or coadministered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, topically, or intrathecally. The compounds and/or compositions according to the invention may also be administered or coadministered in slow release dosage forms.

The modulators and compositions comprising them may be administered or coadministered in any conventional dosage form. Co-administration in the context of this invention is intended to mean the administration of more than one therapeutic agent, one of which includes a kinase inhibitor, in the course of a coordinated treatment to achieve an improved clinical outcome. Such co-administration may also be coextensive, that is, occurring during overlapping periods of time.

Solutions or suspensions used for parenteral, intradermal, subcutaneous, or topical application may optionally include one or more of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; agents for the adjustment of tonicity such as sodium chloride or dextrose, and agents for adjusting the acidity or alkalinity of the composition, such as alkaline or acidifying agents or buffers like carbonates, bicarbonates, phosphates, hydrochloric acid, and organic acids like acetic and citric acid. Parenteral preparations may optionally be enclosed in ampules, disposable syringes or single or multiple dose vials made of glass, plastic or other suitable material.

When modulators according to the present invention exhibit insufficient solubility, methods for solubilizing the compounds may be used. Such methods are known to those of skill in this art, and include, but are not limited to, using cosolvents, such as dimethylsulfoxide (DMSO), using surfactants, such as TWEEN, or dissolution in aqueous sodium bicarbonate. Derivatives of the compounds, such as prodrugs of the compounds may also be used in formulating effective pharmaceutical compositions.

Upon mixing or adding modulators according to the present invention to a composition, a solution, suspension, emulsion or the like may be formed. The form of the resulting composition will depend upon a number of factors, including the intended mode of administration, and the solubility of the compound in the selected carrier or vehicle. The effective concentration needed to ameliorate the disease being treated may be empirically determined.

Compositions according to the present invention are optionally provided for administration to humans and animals in unit dosage forms, such as tablets, capsules, pills, powders, dry powders for inhalers, granules, sterile parenteral solutions or suspensions, and oral solutions or suspensions, and oil-water emulsions containing suitable quantities of the compounds, particularly the pharmaceutically acceptable salts, preferably the sodium salts, thereof. The pharmaceutically therapeutically active compounds and derivatives thereof are typically formulated and administered in unit-dosage forms or multiple-dosage forms. Unit-dose forms, as used herein, refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes individually packaged tablet or capsule. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pint or gallons. Hence, multiple dose form is a multiple of unit-doses that are not segregated in packaging.

In addition to one or more modulators according to the present invention, the composition may comprise: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polyinylpyrrolidine, celluloses and derivatives thereof, povidone, crospovidones and other such binders known to those of skill in the art. Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to form a solution or suspension. If desired, the pharmaceutical composition to be administered may also contain minor amounts of auxiliary substances such as wetting agents, emulsifying agents, or solubilizing agents, pH buffering agents and the like, for example, acetate, sodium citrate, cyclodextrine derivatives, sorbitan monolaurate, triethanolamine sodium acetate, triethanolamine oleate, and other such agents. Actual methods of preparing such dosage forms are known in the art, or will be apparent, to those skilled in this art; (for example, see REMINGTON: THE SCIENCE AND PRACTICE OF PHARMACY, 20^{th} Ed., Gennaro, A. R. (ed.), Mack Publishing Company, Easton, Pa., 2000). The composition or formulation to be administered will, in any event, contain a sufficient quantity of a modulator of the present invention to reduce kinases activity in vivo, thereby treating the disease state of the subject.

### Treatment Regimes

The invention provides pharmaceutical compositions comprising one or a combination of small molecule chemical inhibitors, siRNA inhibitors, or dominant-negative mutants of cyclin dependent kinase activity, e.g., Cdk4 activity (monoclonal, polyclonal or single chain Fv; intact or binding fragments thereof) or nucleic acid compositions, e.g., antisense oligonucleotides, double stranded RNA oligonucleotides (RNAi) or DNA oligonucleotides (vectors) containing nucleotide sequences encoding for the transcription of shRNA molecules, formulated together with a pharmaceutically acceptable carrier. Some compositions include a combination of multiple (e.g., two or more) small chemical molecules, siRNA molecules, monoclonal antibodies or antigen-binding portions thereof of the invention. In some compositions, each of the antibodies or antigen-binding portions thereof of the composition is a monoclonal antibody or a human sequence antibody that binds to a distinct, pre-selected epitope of an antigen.

The pharmaceutical compositions of the invention can be administered in a variety of unit dosage forms depending upon the method of administration. Dosages for the pharmaceutical compositions of the invention are well known to those of skill in the art. Such dosages are typically advisorial in nature and are adjusted depending on the particular therapeutic context, patient tolerance, and the like. The amount of pharmaceutical composition adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age, pharmaceutical formulation and concentration of active agent, and the like. In calculating the dosage regimen for a patient, the mode of administration also is taken into consideration. The dosage regimen must also take into consideration the pharmacokinetics, i.e., the pharmaceutical composition's rate of absorption, bioavailability, metabolism, clearance, and the like.

In therapeutic applications, compositions are administered to a patient suffering from a musculoskeletal disorder to at least partially arrest the condition or a disease and/or its complications. For example, in one aspect, a soluble peptide pharmaceutical composition dosage for intravenous (IV) administration would be about 0.01 mg/hr to about 1.0 mg/hr administered over several hours (typically 1, 3, or 6 hours), which can be repeated for weeks with intermittent cycles. Considerably higher dosages (e.g., ranging up to about 10 mg/ml) can be used, particularly when the drug is administered to a secluded site and not into the blood stream, such as into a body cavity or into a lumen of an organ, e.g., the cerebrospinal fluid (CSF) or a joint space or structure.

In prophylactic applications, pharmaceutical compositions or medicaments are administered to a patient susceptible to, or otherwise at risk of a disease or condition (i.e., an inflammatory-releated disease) in an amount sufficient to eliminate or reduce the risk, lessen the severity, or delay the outset of the disease, including biochemical, histologic and/or behavioral symptoms of the disease, its complications and intermediate pathological phenotypes presenting during development of the disease. In therapeutic applications, compositions or medicants are administered to a patient suspected of, or already suffering from such a disease in an amount sufficient to cure, or at least partially arrest, the symptoms of the disease (biochemical, histologic and/or behavioral), including its complications and intermediate pathological phenotypes in development of the disease. An amount adequate to accomplish therapeutic or prophylactic treatment is defined as a therapeutically- or prophylactically-effective dose. In both prophylactic and therapeutic regimes, agents are usually administered in several dosages until a sufficient immune response has been achieved. Typically, the immune response is monitored and repeated dosages are given if the immune response starts to wane.

“Concomitant administration” of a known drug with a compound of the present invention means administration of the drug and the compound at such time that both the known drug and the compound will have a therapeutic effect or diagnostic effect. Such concomitant administration can involve concurrent (i.e., at the same time), prior, or subsequent administration of the drug with respect to the administration of a compound of the present invention. A person of ordinary skill in the art, would have no difficulty determining the appropriate timing, sequence and dosages of administration for particular drugs and compounds of the present invention.

In general, the phrase “well tolerated” refers to the absence of adverse changes in health status that occur as a result of the treatment and would affect treatment decisions.

### Effective Dosages

Effective doses of the small molecule chemical inhibitors, siRNA inhibitors, or dominant-negative mutants of cyclin dependent kinase activity, e.g., Cdk4 activity, or nucleic acid compositions, e.g., antisense oligonucleotides, double stranded RNA oligonucleotides (RNAi), or DNA oligonucleotides (vectors) containing nucleotide sequences encoding for the transcription of shRNA molecules, for the treatment of inflammatory-related diseases and disorders described herein vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. Treatment dosages need to be titrated to optimize safety and efficacy.

For administration with a small chemical molecule, nucleic acid, siRNA, or antibody composition, the dosage ranges from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once per every two weeks or once a month or once every 3 to 6 months. In some methods, two or more small chemical molecules or siRNA molecules with different binding specificities are administered simultaneously, in which case the dosage of each small chemical molecule, siRNA molecule, or antibody administered falls within the ranges indicated. Small chemical molecule, siRNA molecule, or antibody is usually administered on multiple occasions. Intervals between single dosages can be weekly, monthly or yearly. Intervals can also be irregular as indicated by measuring blood levels of small chemical molecule, siRNA molecule, or antibody in the patient. In some methods, dosage is adjusted to achieve an antibody concentration of 1-1000 μg/ml and in some methods 25-300 μg/ml. Alternatively, antibody can be administered as a sustained release formulation, in which case less frequent administration is required. Dosage and frequency vary depending on the half-life of the compound in the patient. In general, human antibodies show the longest half life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Doses for small chemical molecules, siRNA molecules, or nucleic acids range from about 10 ng to 1 g, 100 ng to 100 mg, 1 μg to 10 mg, or 30-300 μg DNA per patient. Doses for infectious viral vectors vary from 10-100, or more, virions per dose.

Dosage forms or compositions may optionally comprise one or more modulators according to the present invention in the range of 0.005% to 100% (weight/weight) with the balance comprising additional substances such as those described herein. For oral administration, a pharmaceutically acceptable composition may optionally comprise any one or more commonly employed excipients, such as, for example pharmaceutical grades of mannitol, lactose, starch, magnesium stearate, talcum, cellulose derivatives, sodium crosscarmellose, glucose, sucrose, magnesium carbonate, sodium saccharin, talcum. Such compositions include solutions, suspensions, tablets, capsules, powders, dry powders for inhalers and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparing these formulations are known to those skilled in the art. The compositions may optionally contain 0.01%-100% (weight/weight) of one or more modulators, optionally 0.1-95%, and optionally 1-95%.

Salts, preferably sodium salts, of the modulators may be prepared with carriers that protect the compound against rapid elimination from the body, such as time release formulations or coatings. The formulations may further include other active compounds to obtain desired combinations of properties.

### Prodrugs

The present invention is also related to prodrugs of the agents obtained by the methods disclosed herein. Prodrugs are agents which are converted in vivo to active forms (see, e.g., R. B. Silverman, 1992, THE ORGANIC CHEMISTRY OF DRUG DESIGN AND DRUG ACTION, Academic Press, Chp. 8). Prodrugs can be used to alter the biodistribution (e.g., to allow agents which would not typically enter the reactive site of the protease) or the pharmacokinetics for a particular agent. For example, a carboxylic acid group, can be esterified, e.g., with a methyl group or an ethyl group to yield an ester. When the ester is administered to a subject, the ester is cleaved, enzymatically or non-enzymatically, reductively, oxidatively, or hydrolytically, to reveal the anionic group. An anionic group can be esterified with moieties (e.g., acyloxymethyl esters) which are cleaved to reveal an intermediate agent which subsequently decomposes to yield the active agent. The prodrug moieties may be metabolized in vivo by esterases or by other mechanisms to carboxylic acids.

Examples of prodrugs and their uses are well known in the art (see, e.g., Berge et al., “Pharmaceutical Salts”, J. Pharm. Sci. 66: 1-19, 1977). The prodrugs can be prepared in situ during the final isolation and purification of the agents, or by separately reacting the purified agent in its free acid form with a suitable derivatizing agent. Carboxylic acids can be converted into esters via treatment with an alcohol in the presence of a catalyst.

Examples of cleavable carboxylic acid prodrug moieties include substituted and unsubstituted, branched or unbranched lower alkyl ester moieties, (e.g., ethyl esters, propyl esters, butyl esters, pentyl esters, cyclopentyl esters, hexyl esters, cyclohexyl esters), lower alkenyl esters, dilower alkyl-amino lower-alkyl esters (e.g., dimethylaminoethyl ester), acylamino lower alkyl esters, acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, dilower alkyl amides, and hydroxy amides.

### Routes of Administration

Small chemical molecule, siRNA molecule, or antibody compositions for treatment or amelioration of an inflammatory-related disease or disorder, or nucleic acid compositions, e.g., antisense oligonucleotides, double stranded RNA oligonucleotides (RNAi), or DNA oligonucleotides (vectors) containing nucleotide sequences encoding for the transcription of shRNA molecules, for the treatment of an inflammatory-related disease or disorder can be administered by parenteral, topical, intravenous, oral, subcutaneous, intraarterial, intracranial, intraperitoneal, intranasal or intramuscular means for prophylactic as inhalants for small chemical molecule, siRNA molecule or antibody preparations targeting an inflammatory-related disease or disorder, and/or therapeutic treatment. The most typical route of administration of an immunogenic agent is subcutaneous although other routes can be equally effective. The next most common route is intramuscular injection. This type of injection is most typically performed in the arm or leg muscles. In some methods, agents are injected directly into a particular tissue where a tumor is found, for example intracranial injection or convection enhanced delivery. Intramuscular injection or intravenous infusion are preferred for administration of antibody. In some methods, particular therapeutic antibodies are delivered directly into the cranium. In some methods, antibodies are administered as a sustained release composition or device, such as a Mediapad™ device.

Agents of the invention can optionally be administered in combination with other agents that are at least partly effective in treating various diseases including various immune-related diseases. In the case of infection in the brain, agents of the invention can also be administered in conjunction with other agents that increase passage of the agents of the invention across the blood-brain barrier (BBB).

### Formulation

Modulators of the present invention, such as small chemical molecule, siRNA molecule, or antibody inhibitors of cyclin dependent kinase, nucleic acid compositions, e.g., antisense oligonucleotides, double stranded RNA oligonucleotides (RNAi), or DNA oligonucleotides (vectors) containing nucleotide sequences encoding for the transcription of shRNA molecules, for the treatment of an inflammatory-related disease or disorder, are often administered as pharmaceutical compositions comprising an active therapeutic agent, i.e., and a variety of other pharmaceutically acceptable components (see, e.g., REMINGTON: THE SCIENCE AND PRACTICE OF PHARMACY, 20^{th} Ed., Gennaro, A. R. (ed.), Mack Publishing Company, Easton, Pa., 2000, incorporated herein by reference). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

For parenteral administration, compositions of the invention can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies can be administered in the form of a depot injection or implant preparation which can be formulated in such a manner as to permit a sustained release of the active ingredient. An exemplary composition comprises monoclonal antibody at 5 mg/mL, formulated in aqueous buffer consisting of 50 mM L-histidine, 150 mM NaCl, adjusted to pH 6.0 with HCl.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990; Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%.

A. Formulations for Oral Administration

Oral pharmaceutical dosage forms may be as a solid, gel or liquid. Examples of solid dosage forms include, but are not limited to tablets, capsules, granules, and bulk powders. More specific examples of oral tablets include compressed, chewable lozenges and tablets that may be enteric-coated, sugar-coated or film-coated. Examples of capsules include hard or soft gelatin capsules. Granules and powders may be provided in non-effervescent or effervescent forms. Each may be combined with other ingredients known to those skilled in the art.

Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

In certain aspects, modulators according to the present invention are provided as solid dosage forms, preferably capsules or tablets. The tablets, pills, capsules, troches and the like may optionally contain one or more of the following ingredients, or compounds of a similar nature: a binder; a diluent; a disintegrating agent; a lubricant; a glidant; a sweetening agent; and a flavoring agent.

Examples of binders that may be used include, but are not limited to, microcrystalline cellulose, gum tragacanth, glucose solution, acacia mucilage, gelatin solution, sucrose and starch paste.

Examples of lubricants that may be used include, but are not limited to, talc, starch, magnesium or calcium stearate, lycopodium and stearic acid.

Examples of diluents that may be used include, but are not limited to, lactose, sucrose, starch, kaolin, salt, mannitol and dicalcium phosphate.

Examples of glidants that may be used include, but are not limited to, colloidal silicon dioxide.

Examples of disintegrating agents that may be used include, but are not limited to, crosscarmellose sodium, sodium starch glycolate, alginic acid, corn starch, potato starch, bentonite, methylcellulose, agar and carboxymethylcellulose.

Examples of coloring agents that may be used include, but are not limited to, any of the approved certified water soluble FD and C dyes, mixtures thereof; and water insoluble FD and C dyes suspended on alumina hydrate.

Examples of sweetening agents that may be used include, but are not limited to, sucrose, lactose, mannitol and artificial sweetening agents such as sodium cyclamate and saccharin, and any number of spray-dried flavors.

Examples of flavoring agents that may be used include, but are not limited to, natural flavors extracted from plants such as fruits and synthetic blends of compounds that produce a pleasant sensation, such as, but not limited to peppermint and methyl salicylate.

Examples of wetting agents that may be used include, but are not limited to, propylene glycol monostearate, sorbitan monooleate, diethylene glycol monolaurate and polyoxyethylene lauryl ether.

Examples of anti-emetic coatings that may be used include, but are not limited to, fatty acids, fats, waxes, shellac, ammoniated shellac and cellulose acetate phthalates.

Examples of film coatings that may be used include, but are not limited to, hydroxyethylcellulose, sodium carboxymethylcellulose, polyethylene glycol 4000 and cellulose acetate phthalate.

If oral administration is desired, the salt of the compound may optionally be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it may optionally additionally comprise a liquid carrier such as a fatty oil. In addition, dosage unit forms may optionally additionally comprise various other materials that modify the physical form of the dosage unit, for example, coatings of sugar and other enteric agents.

Compounds according to the present invention may also be administered as a component of an elixir, suspension, syrup, wafer, sprinkle, chewing gum or the like. A syrup may optionally comprise, in addition to the active compounds, sucrose as a sweetening agent and certain preservatives, dyes and colorings and flavors.

The modulators of the present invention may also be mixed with other active materials that do not impair the desired action, or with materials that supplement the desired action, such as antacids, H2 blockers, and diuretics. For example, if a compound is used for treating asthma or hypertension, it may be used with other bronchodilators and antihypertensive agents, respectively.

Examples of pharmaceutically acceptable carriers that may be included in tablets comprising modulators of the present invention include, but are not limited to binders, lubricants, diluents, disintegrating agents, coloring agents, flavoring agents, and wetting agents. Enteric-coated tablets, because of the enteric-coating, resist the action of stomach acid and dissolve or disintegrate in the neutral or alkaline intestines. Sugar-coated tablets may be compressed tablets to which different layers of pharmaceutically acceptable substances are applied. Film-coated tablets may be compressed tablets that have been coated with polymers or other suitable coating. Multiple compressed tablets may be compressed tablets made by more than one compression cycle utilizing the pharmaceutically acceptable substances previously mentioned. Coloring agents may also be used in tablets. Flavoring and sweetening agents may be used in tablets, and are especially useful in the formation of chewable tablets and lozenges.

Examples of liquid oral dosage forms that may be used include, but are not limited to, aqueous solutions, emulsions, suspensions, solutions and/or suspensions reconstituted from non-effervescent granules and effervescent preparations reconstituted from effervescent granules.

Examples of aqueous solutions that may be used include, but are not limited to, elixirs and syrups. As used herein, elixirs refer to clear, sweetened, hydroalcoholic preparations. Examples of pharmaceutically acceptable carriers that may be used in elixirs include, but are not limited to solvents. Particular examples of solvents that may be used include glycerin, sorbitol, ethyl alcohol and syrup. As used herein, syrups refer to concentrated aqueous solutions of a sugar, for example, sucrose. Syrups may optionally further comprise a preservative.

Emulsions refer to two-phase systems in which one liquid is dispersed in the form of small globules throughout another liquid. Emulsions may optionally be oil-in-water or water-in-oil emulsions. Examples of pharmaceutically acceptable carriers that may be used in emulsions include, but are not limited to non-aqueous liquids, emulsifying agents and preservatives.

Examples of pharmaceutically acceptable substances that may be used in non-effervescent granules, to be reconstituted into a liquid oral dosage form, include diluents, sweeteners and wetting agents.

Examples of pharmaceutically acceptable substances that may be used in effervescent granules, to be reconstituted into a liquid oral dosage form, include organic acids and a source of carbon dioxide. Coloring and flavoring agents may optionally be used in all of the above dosage forms.

Particular examples of preservatives that may be used include glycerin, methyl and propylparaben, benzoic add, sodium benzoate and alcohol. Particular examples of non-aqueous liquids that may be used in emulsions include mineral oil and cottonseed oil. Particular examples of emulsifying agents that may be used include gelatin, acacia, tragacanth, bentonite, and surfactants such as polyoxyethylene sorbitan monooleate. Particular examples of suspending agents that may be used include sodium carboxymethylcellulose, pectin, tragacanth, Veegum and acacia. Diluents include lactose and sucrose. Sweetening agents include sucrose, syrups, glycerin and artificial sweetening agents such as sodium cyclamate and saccharin. Particular examples of wetting agents that may be used include propylene glycol monostearate, sorbitan monooleate, diethylene glycol monolaurate and polyoxyethylene lauryl ether. Particular examples of organic acids that may be used include citric and tartaric acid.

Sources of carbon dioxide that may be used in effervescent compositions include sodium bicarbonate and sodium carbonate. Coloring agents include any of the approved certified water soluble FD and C dyes, and mixtures thereof.

Particular examples of flavoring agents that may be used include natural flavors extracted from plants such fruits, and synthetic blends of compounds that produce a pleasant taste sensation.

For a solid dosage form, the solution or suspension, in for example propylene carbonate, vegetable oils or triglycerides, is preferably encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. Re 28,819 and 4,358,603.

B. Injectables, Solutions, and Emulsions

The present invention is also directed to compositions designed to administer the modulators of the present invention by parenteral administration, generally characterized by injection, either subcutaneously, intramuscularly or intravenously. Injectables may be prepared in any conventional form, for example as liquid solutions or suspensions, solid forms suitable for solution or suspension in liquid prior to injection, or as emulsions.

Examples of excipients that may be used in conjunction with injectables according to the present invention include, but are not limited to water, saline, dextrose, glycerol or ethanol. The injectable compositions may also optionally comprise minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained (see, e.g., U.S. Pat. No. 3,710,795) is also contemplated herein. The percentage of active compound contained in such parenteral compositions is highly dependent on the specific nature thereof, as well as the activity of the compound and the needs of the subject.

Parenteral administration of the formulations includes intravenous, subcutaneous and intramuscular administrations. Preparations for parenteral administration include sterile solutions ready for injection, sterile dry soluble products, such as the lyophilized powders described herein, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

When administered intravenously, examples of suitable carriers include, but are not limited to physiological saline or phosphate buffered saline (PBS), and solutions containing thickening and solubilizing agents, such as glucose, polyethylene glycol, and polypropylene glycol and mixtures thereof.

Examples of pharmaceutically acceptable carriers that may optionally be used in parenteral preparations include, but are not limited to aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles that may optionally be used include Sodium Chloride Injection, Ringers Injection, Isotonic Dextrose Injection, Sterile Water Injection, Dextrose and Lactated Ringers Injection.

Examples of nonaqueous parenteral vehicles that may optionally be used include fixed oils of vegetable origin, cottonseed oil, corn oil, sesame oil and peanut oil.

Antimicrobial agents in bacteriostatic or fungistatic concentrations may be added to parenteral preparations, particularly when the preparations are packaged in multiple-dose containers and thus designed to be stored and multiple aliquots to be removed. Examples of antimicrobial agents that may be used include phenols or cresols, mercurials, benzyl alcohol, chlorobutanol, methyl and propyl p-hydroxybenzoic acid esters, thimerosal, benzalkonium chloride and benzethonium chloride.

Examples of isotonic agents that may be used include sodium chloride and dextrose. Examples of buffers that may be used include phosphate and citrate. Examples of antioxidants that may be used include sodium bisulfate. Examples of local anesthetics that may be used include procaine hydrochloride. Examples of suspending and dispersing agents that may be used include sodium carboxymethylcellulose, hydroxypropyl methylcellulose and polyvinylpyrrolidone. Examples of emulsifying agents that may be used include Polysorbate 80 (TWEEN 800). A sequestering or chelating agent of metal ions include EDTA.

Pharmaceutical carriers may also optionally include ethyl alcohol, polyethylene glycol and propylene glycol for water miscible vehicles and sodium hydroxide, hydrochloric acid, citric acid or lactic acid for pH adjustment.

The concentration of a modulator in the parenteral formulation may be adjusted so that an injection administers a pharmaceutically effective amount sufficient to produce the desired pharmacological effect. The exact concentration of a modulator and/or dosage to be used will ultimately depend on the age, weight and condition of the patient or animal as is known in the art.

Unit-dose parenteral preparations may be packaged in an ampoule, a vial or a syringe with a needle. All preparations for parenteral administration should be sterile, as is know and practiced in the art.

Injectables may be designed for local and systemic administration. Typically a therapeutically effective dosage is formulated to contain a concentration of at least about 0.1% w/w up to about 90% w/w or more, preferably more than 1% w/w of the modulator to the treated tissue(s). The modulator may be administered at once, or may be divided into a number of smaller doses to be administered at intervals of time. It is understood that the precise dosage and duration of treatment will be a function of the location of where the composition is parenterally administered, the carrier and other variables that may be determined empirically using known testing protocols or by extrapolation from in vivo or in vitro test data. It is to be noted that concentrations and dosage values may also vary with the age of the individual treated. It is to be further understood that for any particular subject, specific dosage regimens may need to be adjusted over time according to the individual need and the professional judgment of the person administering or supervising the administration of the formulations. Hence, the concentration ranges set forth herein are intended to be exemplary and are not intended to limit the scope or practice of the claimed formulations.

The modulator may optionally be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the disease state and may be empirically determined.

C. Lyophilized Powders

The modulators of the present invention may also be prepared as lyophilized powders, which can be reconstituted for administration as solutions, emulsions and other mixtures. The lyophilized powders may also be formulated as solids or gels.

Sterile, lyophilized powder may be prepared by dissolving the compound in a sodium phosphate buffer solution containing dextrose or other suitable excipient. Subsequent sterile filtration of the solution followed by lyophilization under standard conditions known to those of skill in the art provides the desired formulation. Briefly, the lyophilized powder may optionally be prepared by dissolving dextrose, sorbitol, fructose, corn syrup, xylitol, glycerin, glucose, sucrose or other suitable agent, about 1-20%, preferably about 5 to 15%, in a suitable buffer, such as citrate, sodium or potassium phosphate or other such buffer known to those of skill in the art at, typically, about neutral pH. Then, a modulator is added to the resulting mixture, preferably above room temperature, more preferably at about 30-35° C., and stirred until it dissolves. The resulting mixture is diluted by adding more buffer to a desired concentration. The resulting mixture is sterile filtered or treated to remove particulates and to insure sterility, and apportioned into vials for lyophilization. Each vial may contain a single dosage or multiple dosages of the modulator.

D. Topical Administration

The modulators of the present invention may also be administered as topical mixtures. Topical mixtures may be used for local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

Topical application can result in transdermal or intradermal delivery. Topical administration can be facilitated by co-administration of the agent with cholera toxin or detoxified derivatives or subunits thereof or other similar bacterial toxins. Glenn et al., Nature 391: 851, 1998. Co-administration can be achieved by using the components as a mixture or as linked molecules obtained by chemical crosslinking or expression as a fusion protein.

Alternatively, transdermal delivery can be achieved using a skin patch or using transferosomes. Paul et al., Eur. J. Immunol. 25: 3521-24, 1995; Cevc et al., Biochem. Biophys. Acta 1368: 201-15, 1998.

The modulators may be formulated as aerosols for topical application, such as by inhalation (see, U.S. Pat. Nos. 4,044,126, 4,414,209, and 4,364,923, which describe aerosols for delivery of a steroid useful for treatment inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will typically have diameters of less than 50 microns, preferably less than 10 microns.

The modulators may also be formulated for local or topical application, such as for topical application to the skin and mucous membranes, such as in the eye, in the form of gels, creams, and lotions and for application to the eye or for intracistemal or intraspinal application. Topical administration is contemplated for transdermal delivery and also for administration to the eyes or mucosa, or for inhalation therapies. Nasal solutions of the modulator alone or in combination with other pharmaceutically acceptable excipients can also be administered.

The pharmaceutical compositions are generally formulated as sterile, substantially isotonic and in full compliance with all Good Manufacturing Practice (GMP) regulations of the U.S. Food and Drug Administration.

### Toxicity

Preferably, a therapeutically effective dose of the small chemical molecule, siRNA molecule, antibody, or nucleic acid compositions, e.g., antisense oligonucleotides, double stranded RNA oligonucleotides (RNAi), or DNA oligonucleotides (vectors) containing nucleotide sequences encoding for the transcription of shRNA molecules, described herein will provide therapeutic benefit without causing substantial toxicity.

Toxicity of the proteins described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition (see, e.g., Fingl et al., 1975, In: THE PHARMACOLOGICAL BASIS OF THERAPEUTICS, Ch. 1,

### Kits

The invention is also directed to kits and other articles of manufacture for treating inflammatory-releated diseases and disorders associated with kinases. It is noted that diseases are intended to cover all conditions for which the kinases possesses activity that contributes to the pathology and/or symptomology of the condition.

In one aspect, a kit is provided that comprises a composition comprising at least one small chemical molecule, siRNA molecule, antibody, or nucleic acid compositions, e.g., antisense oligonucleotides, double stranded RNA oligonucleotides (RNAi), or DNA oligonucleotides (vectors) containing nucleotide sequences encoding for the transcription of shRNA molecules) of the present invention in combination with instructions. The instructions may indicate the disease state for which the composition is to be administered, storage information, dosing information and/or instructions regarding how to administer the composition. The kit may also comprise packaging materials. The packaging material may comprise a container for housing the composition. The kit may also optionally comprise additional components, such as syringes for administration of the composition. The kit may comprise the composition in single or multiple dose forms.

In another aspect, an article of manufacture is provided that comprises a composition comprising at least one kinase inhibitor of the present invention in combination with packaging materials. The packaging material may comprise a container for housing the composition. The container may optionally comprise a label indicating the disease state for which the composition is to be administered, storage information, dosing information and/or instructions regarding how to administer the composition. The kit may also optionally comprise additional components, such as syringes for administration of the composition. The kit may comprise the composition in single or multiple dose forms.

It is noted that the packaging material used in kits and articles of manufacture according to the present invention may form a plurality of divided containers such as a divided bottle or a divided foil packet. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container that is employed will depend on the exact dosage form involved, for example a conventional cardboard box would not generally be used to hold a liquid suspension. It is feasible that more than one container can be used together in a single package to market a single dosage form. For example, tablets may be contained in a bottle that is in turn contained within a box. Typically the kit includes directions for the administration of the separate components. The kit form is particularly advantageous when the separate components are preferably administered in different dosage forms (e.g., oral, topical, transdermal and parenteral), are administered at different dosage intervals, or when titration of the individual components of the combination is desired by the prescribing physician.

One particular example of a kit according to the present invention is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

Another specific aspect of a kit is a dispenser designed to dispense the daily doses one at a time in the order of their intended use. Preferably, the dispenser is equipped with a memory-aid, so as to further facilitate compliance with the regimen. An example of such a memory-aid is a mechanical counter that indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

Each recited range includes all combinations and sub-combinations of ranges, as well as specific numerals contained therein.

All publications and patent applications cited in this specification are herein incorporated by reference in their entirety for all purposes as if each individual publication or patent application were specifically and individually indicated to be incorporated by reference for all purposes.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

The following Exemplary Embodiments of specific embodiments for carrying out the present invention are offered for illustrative purposes only, and are not intended to limit the scope of the present invention in any way.

### Exemplary Embodiments

### Materials and Methods

Cells. Jurkat cells and Ramos B cells were obtained from the American Type Culture Collection (ATCC) and were cultured in RPMI (Mediatech, Herndon Va.) supplemented with glutaMAX-1 (Invitrogen, Carlsbad Calif.), 1 mM sodium pyruvate (BioWhittaker, Walkersville Md.), nonessential amino acids (NEAA) (BioWhittaker) and 10% fetal bovine serum (FBS) (Hyclone, Logan Utah). Peripheral blood was obtained from healthy donors with informed consent according to protocols approved by the Human Subjects Review Committee of the University of Washington. PBMC were isolated by Ficoll-Hypaque (Pharmacia, Piscataway, N.J.) gradient centrifugation and washed with PBS. HUVEC were isolated and cultured as previously described (Schwartz et al., J Immunol 162: 4842-8, 1999) and were grown in RPMI 1640 with supplemented with 2 mM glutamine, sodium pyruvate, NEAA, 10 mM HEPES, 100 U/ml penicillin, 100 U/ml streptomycin, 250 ng/ml fungizone (BioWhittaker), 90 mg/ml heparin (Sigma, St. Louis, Mo.), bovine hypothalamic extract (gift of E. Raines, University of Washington, Seattle, Wash.), and 10% bovine calf serum supplemented with iron (HyClone). HUVEC were cultured on surfaces coated with 2% gelatin (Sigma). BAEC were a gift of Helene Sage (Hope Heart Institute, Seattle Wash.) and were grown in DMEM (Mediatech) supplemented with glutaMAX-1, sodium pyruvate and 8% FBS. EC matrix was prepared by lysis of a confluent layer of EC with 20 mM NH₄OH at 37° C. for 5 min (Kato and Gospodarowicz, J Cell Biol 100: 486-95, 1985) and blocked briefly with growth medium. Adhesion assays. Lymphocytes were labeled with 2.5 mM calcein-AM (Molecular Probes, Eugene, Oreg.) at room temperature for 20-40 min, washed, and resuspended in HBSS with calcium and magnesium (Mediatech) supplemented with 0.1% BSAnd 4 mM HEPES. Lymphocytes were incubated with inhibitors or function-blocking antibodies at 37° C. for 15-30 min and tested for LIA or phorbol ester-stimulated adhesion to EC or to EC matrix for 15-30 min at 37° C. Adhesion was measured by a Cytofluor Series 4000 fluorescence plate reader (PerSeptive Biosystems, Framingham, Mass.). Plates were scanned before and after washing for total and adherent cells, respectively. Statistical significance was deteremined by Student's t-Test. In some cases, EC were labeled with Cell Tracker Orange (Molecular Probes) per manufacturers instructions prior to adhesion. Cells were fixed after adhesion assays and adherent cells were directly visualized by fluorescent microscopy. Underlying fibronectin fibrils were visualized with polyclonal anti-bovine fibronectin antibody (Accurate), followed by Alexa-568 conjugated secondary antibody (Molecular Probes).

Transfections. Cdk2 and Cdk4 dominant negative constructs were the generous gift of James Roberts (Fred Hutchinson Cancer Research Center, Seattle, Wash.). van den Heuvel and Harlow, Science 262: 2050-4, 1993. Phosphorylation-deficient construct of pRb was a gift from Lukas Lukas et al., Genes Dev 11: 1479-92, 1997; Lukas et al., Oncogene 18: 3930-5, 1999. Transfections were performed with TransIT-Jurkat Transfection Reagent (Mirus, Gene Transfer Specialist™) according to the manufacturer's instructions. Controls consisted of cells transfected with empty vector pCMV alone. Stably transfected cell lines were selected in medium containing the neomycin analog G418 (1 mg/ml). Expression was verified by Western blot analysis. Generation of Rap 1 dominant negative transfectants was previously described. Liu et al., J Biol Chem 277: 40893-900, 2002. Cdk4 siRNA was purchased from Invitrogen and transient transfections were performed by electroporation using the Amaxa Biosystems . . . Western Blot Analysis. Equal numbers of Jurkat cells (1×10⁶) were lysed in buffer containing 50 mM Hepes, pH 7.5, 10 mM NaCl, 5 mM MgCl₂, 1 mM EGTA, 1 μg/ml leupeptin, 1 μg/ml aprotinin, 1% triton, and 10% glycerol and Complete Mini Protease Inhibitor Cocktail (Roche). Equal amounts of protein were separated by SDS-PAGE, transferred to Immobilon and blocked for 1 hour. Blots were incubated with primary antibody for 1 hour, followed by peroxidase-conjugated secondary antibody for 1 hour and then developed with Enhanced Chemiluminescence (ECL, Amersham). Anti-phosphorylated Rb (T821) and anti-phosphorylated Rb (249/252) were obtained from Biosource. Anti-Cdk2 and anti-Cdk4 antibodies were obtained from eBioscience.

Reagants/antibodies. Cdk inhibitors aminopurvalanol A, purvalanol And roscovitine were obtained from Tocris Bioscience (Ellisville, Mo.). Cytochalasin D and additional inhibitors were obtained from Calbiochem. Function blocking antibodies to α4 and α5 integrin subunits were obtained from Chemicon. β integrin blocking antibody 5D1 was previously characterized. van den Heuvel and Harlow, Science 262: 2050-4, 1993. The β1 integrin antibody 9EG7, which recognizes the activation epitope, (Lenter et al., Proceedings Of The National Academy Of Sciences Of The United States Of America 90: 9051-9055, 1993) was obtained from BD Biosciences. All antibodies were used at saturating concentrations.

Flow cytometry. Integrin subunit expression levels were analyzed by a FACScan (Becton Dickinson) instrument. Analyzed cells were incubated in integrin antibodies for 30 min, followed by incubation with fluorescein-labeled secondary antibodies for 30 mins. Migration Assays. Calcein-labeled PBMC were treated with purvanalol for 15 min, then added to a confluent monolayer of HUVEC grown on collagen-coated transwell (3 micron pore size, Costar-Corning). PBMC were allowed to migrate for 4 hours at 37° C. Number of migrated cells in bottom chamber was counted. Treatment of PBMC with purvanol did not affect initial adhesion to confluent endothelial cells. In addition, in some experiments, equal numbers of PBMC were allowed to adhere to HUVEC, then treated with purvanaol, to verify that differences in migration were not simply due to differences in initial adhesion. All assays were performed in triplicate. Integrity of the endothelial monolayer was verified by measurements of transendothelial resistance before and after migration assay.

### Results

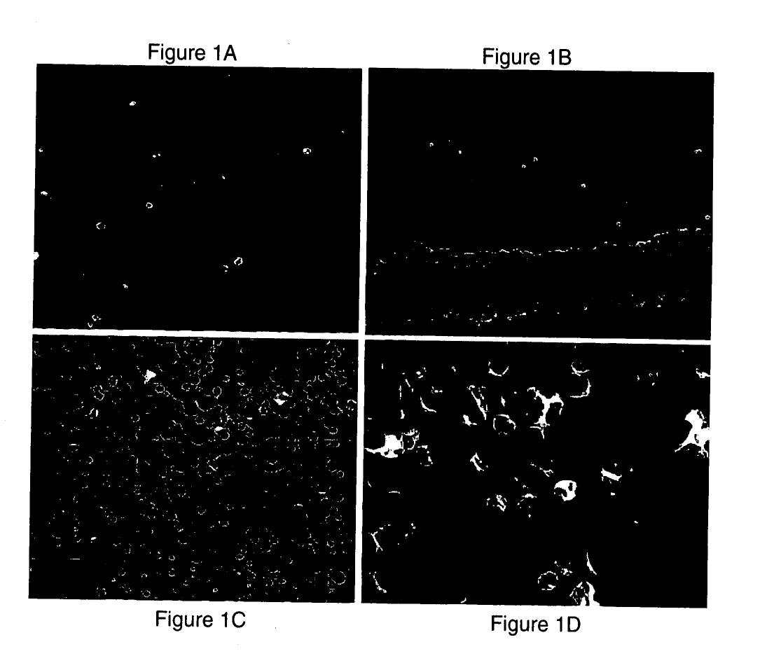

Lymphocytes adhere spontaneously to underlying endothelial cell matrix. Confluent bovine aortic endothelial cells (BAEC) or human umbilical vein endothelial cells (HUVEC) were treated with low-dose cytochalasin D to cause them to retract. Untreated Jurkat cells were allowed to adhere to cytochalasin-treated endothelial cells (EC). We found minimal Jurkat cell adhesion to intact endothelial monolayers (FIG. 1A), but large numbers of Jurkat cells around retracted endothelial cells (FIG. 1C). In addition, wounding an endothelial monolayer by scratching also caused Jurkat cells to adhere spontaneously to the margin of the retracted monolayer (FIG. 1B). When endothelial cells were removed by NH₄OH treatment, the remaining underlying matrix also supported spontaneous lymphocyte adhesion (FIG. 1D). Similar results were found using freshly isolated peripheral blood mononuclear cells (PBMC).

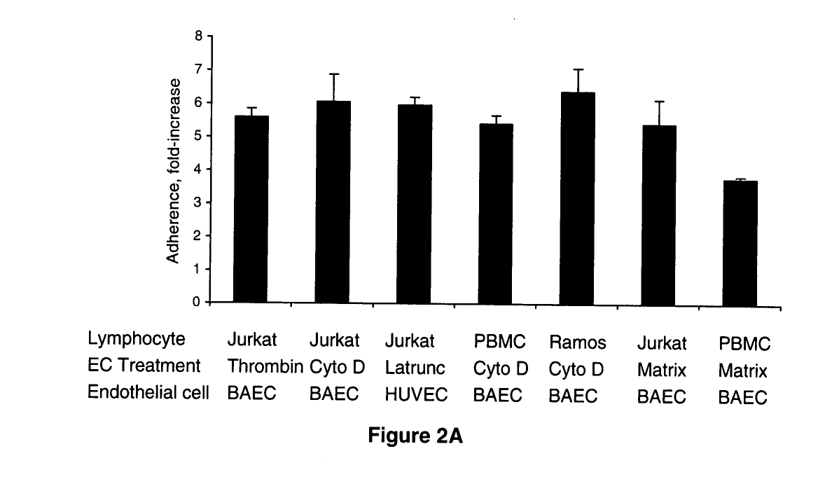

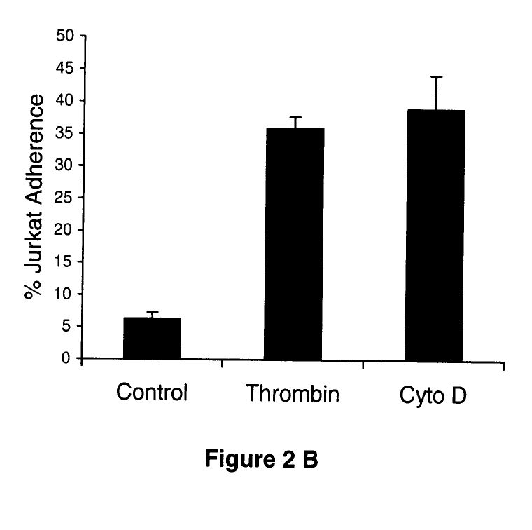

To quantify adhesion, we performed cell adhesion assays as previously described. Liu et al., J Biol Chem 277: 40893-900, 2002. PBMC adhesion to cytochalasin-treated EC was 5-6 fold greater than PBMC adhesion to untreated EC (FIG. 2). PBMC adhesion to underlying EC matrix was also 5-6 fold greater than adhesion to EC monolayer. Similar results were found with Jurkat cells and Ramos B cells. To examine a more physiological agent, we treated BAEC with thrombin. Thrombin, like cytochalasin D, caused endothelial cell retraction and increased Jurkat cell adhesion by 4-5 fold (FIG. 2B). Additional reagents that caused endothelial cell retraction, including staurosporine, latrunculin, EDTA, and PBS without calcium and magnesium, showed similar enhancement of adhesion (FIG. 2A). Of note, treatment of EC with agents that induced apoptosis but failed to induce retraction, such as colchicine, vinblastine, nocadozole, and paclitaxel, also failed to produce comparable lymphocyte adhesion (not shown). Similar results were found whether BAEC or HUVEC were used.

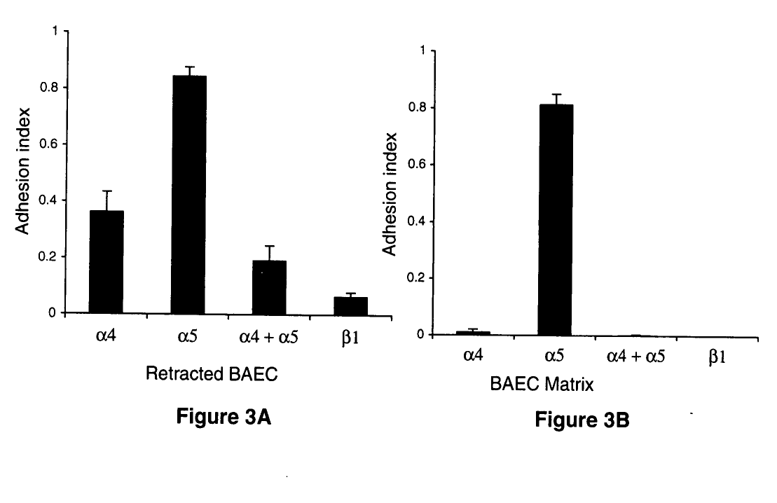

Integrins mediate binding to many components of endothelial matrix. We previously reported that increased Jurkat adhesion to EC treated with staurosporine was β1 integrin-dependent. To determine the cell adhesion receptors involved in LIA, we pretreated Jurkat cells with function blocking antibodies to β1, α4 and α5 integrin subunits. Pretreatment with β1 blocking antibody almost completely abrogated adhesion to retracted EC or EC matrix (FIG. 3A, B). Pretreatment with α4 blocking antibody also significantly decreased cell adhesion. In contrast, blockade of α5 had no significant effect on adhesion to retracted EC or EC matrix, although blockade of both α4 and α5 further reduced cell adhesion to retracted EC. The results suggest that α4β1 is the primary integrin involved in LIA of lymphocytes to EC matrix. α5β1 may contribute to LIA when α4β1 is not available.

Spontaneous adhesion to matrix is not dependent on Rap1. We previously showed that phorbol ester-stimulated Jurkat adhesion to low density FN was dependent on Rap1A activity. Liu et al., J Biol Chem 277: 40893-900, 2002. To determine the contribution of Rap1 to LIA, we stably transfected Jurkat cells with a dominant negative construct of Rap1, N17Rap1. In contrast to phorbol ester-stimulated adhesion, overexpression of the dominant negative Rap1 had no effect on LIA of Jurkat cells to BAEC-derived matrix. We also previously showed that overexpression of the Rap1-selective GTPase-activating protein SPA, which keeps Rap1 in its inactive GDP form, had no effect on spontaneous adhesion of Jurkat cells to high-density FN. Liu et al., J Biol Chem 277: 40893-900, 2002. We now extend these observations to adhesion to EC matrix and find that it is similarly Rap1-independent.

We previously reported that moderate dose (1 μM) cytochalasin D treatment of Jurkat cells inhibited phorbol ester-stimulated cell adhesion. In contrast, the same dose of cytochalasin had no affect on ligand-induced cell adhesion.

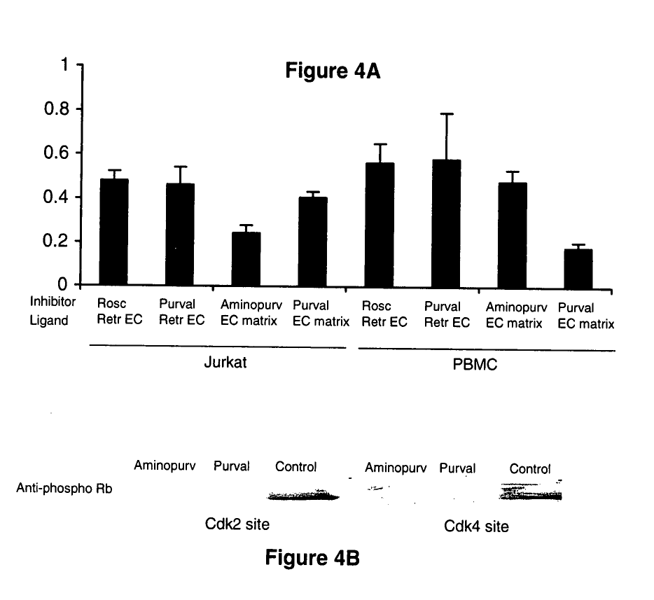

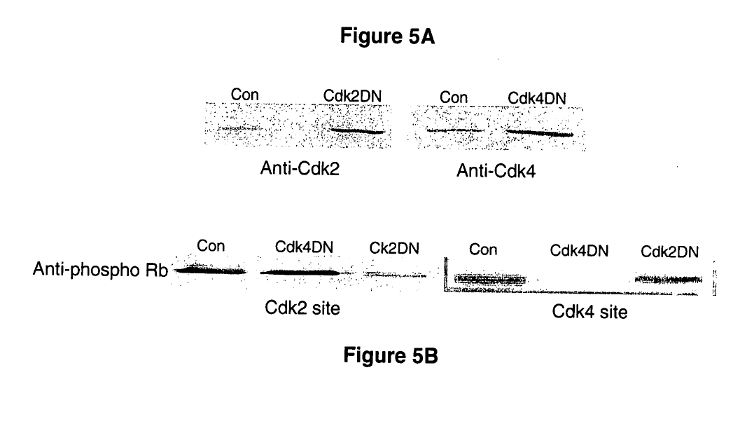

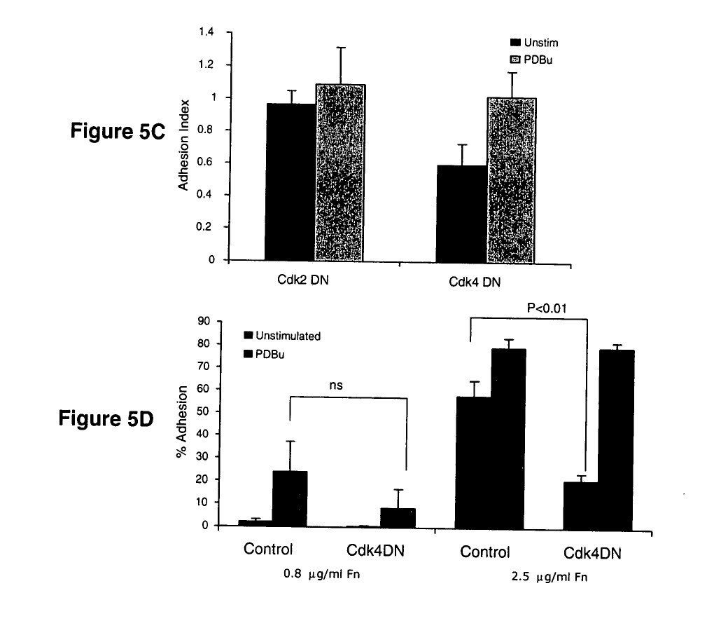

Pharmacological inhibitors of Cdk, but not other kinases inhibit spontaneous adhesion to matrix. To determine the pathway(s) involved in LIA, we tested the effects of pharmacological inhibitors of various pathways on LIA. Only Cdk inhibitors blocked LIA (FIG. 5A). When Jurkat or PBMC were treated with the Cdk inhibitors roscovitine, purvalanol A, or aminopurvalanol A, their adhesion to retracted BAEC or EC matrix was significantly reduced (FIG. 5A). We previously showed that lymphocytes were able to adhere spontaneously to high density fibronectin in a Rap-1 independent manner. We now show that spontaneous adhesion to high density fibronectin is also inhibited by Cdk inhibitor (FIG. 4B). In contrast, the Cdk inhibitors had no effect on phorbol ester-stimulated adhesion (not shown). Inhibitors of other pathways, including MAPK (PD98059), RhoA kinase (Y-7632), PI3 kinase (LY294002) and tyrosine kinases (genistein) did not inhibit LIA.

To verify the efficacy of the Cdk inhibitors, we examined phosphorylation of retinoblastoma protein (Rb), the major target of Cdk. Cdk2 and Cdk4 phosphorylate Rb at different sites: Cdk2 phosphorylation sites include threonine 821, and Cdk4 phosphorylation sites include serine 249 and threonine 252. Mittnacht, Curr Opin Genet Dev 8: 21-7, 1998. We confirmed that treatment with the Cdk inhibitors blocked phosphorylation of Rb using phospho-specific antibodies (FIG. 4C). Of note, despite the reported specificity of roscovitine and aminopurvalanol A for Cdk2, and not Cdk4 (Knockaert et al., Trends Pharmacol Sci. 23: 417-25, 2002a), we found that the inhibitors blocked phosphorylation of both Cdk sites. Thus, the Cdk inhibitors tested function as broad-spectrum Cdk inhibitors, rather than subtype-specific inhibitors. Nevertheless, these data suggested that Cdks might be involved in LIA.

Spontaneous adhesion to matrix is inhibited by dominant negative construct of Cdk4, but not Cdk2. Recent studies have shown that Cdk inhibitors can target additional pathways, including MAPK. Knockaert et al., Oncogene 21: 6413-24, 2002b. Therefore, to confirm our studies using pharmacological inhibitors, and to determine which Cdk was involved in ligand-induced adhesion, we tested the ability of Cdk dominant-negative (DN) constructs to inhibit LIA. We stably transfected Jurkat cells with a Cdk4 expression vector or Cdk2 expression vector that contained a mutation D145N that prevents their activity. van den Heuvel and Harlow, Science 262: 2050-4, 1993. We confirmed expression of the constructs by Western blot analysis (FIG. 5A). Since Cdk activity is required for cell proliferation, we were able to select only low-moderate expressing cells, but were able to achieve >50% inhibition of phosphorylation at their specific sites in Rb (FIG. 5B). Interestingly, only Cdk4 DN reduced the adhesion; Cdk2 DN did not inhibit LIA (FIG. 5C). Importantly, neither construct affected phorbol ester-stimulated adhesion. These results show that Cdks, specifically Cdk4, are involved in ligand-induced cell adhesion.

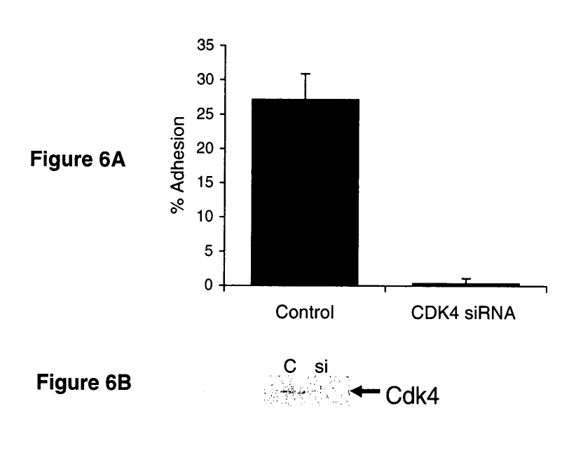

Cdk4 siRNA inhibits LIA. To further confirm the role of Cdk4 in LIA, we examined the effect of Cdk siRNA on LIA. Maximal reduction in Cdk4 protein was achieved at 48 hours after initial transfection, as indicated by Western blot analysis (FIG. 6A). At that time, LIA was almost completely abolished (FIG. 6B). Control vector had no effect.

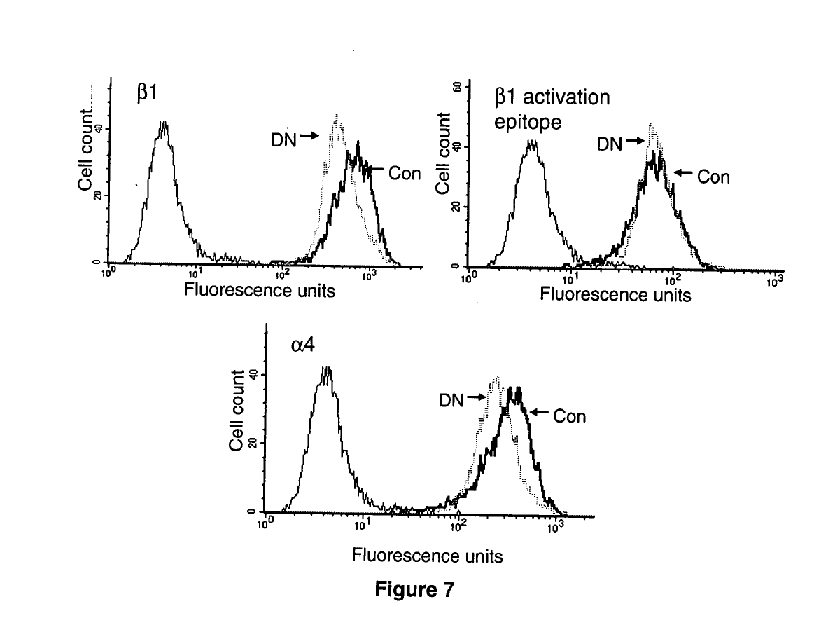

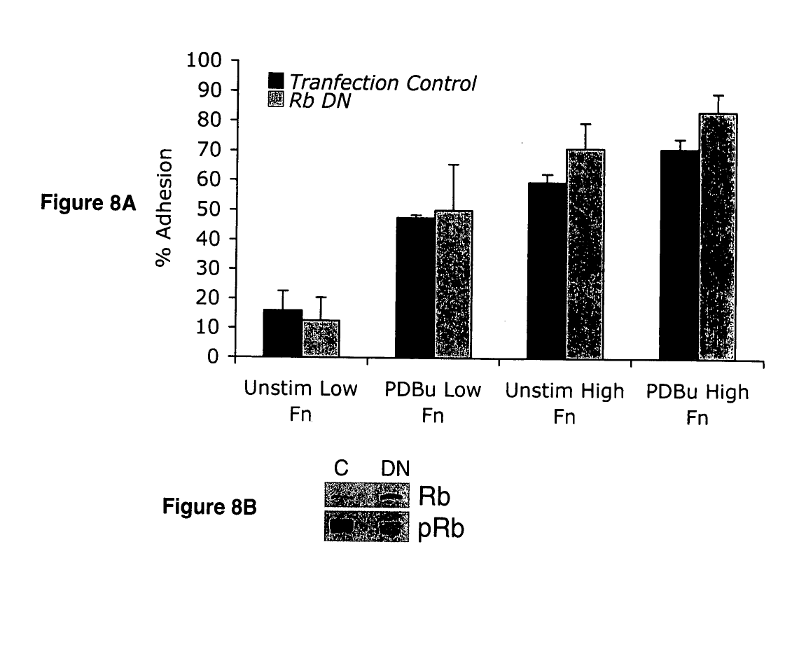

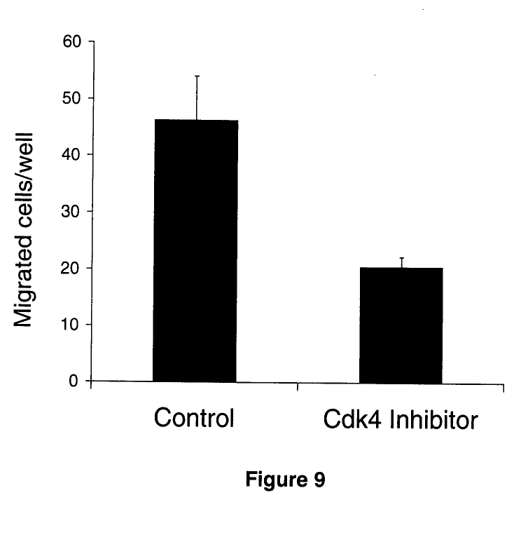

No change in integrin expression or conformation following Cdk4 blockade. Because LIA is spontaneous, and Cdk inhibitors rapidly (within 20 minutes) inhibit LIA, it is unlikely to involve differences in surface expression of β₁ integrin. However, integrins can exist in different conformational states, and many integrins exist at rest in a low affinity state. Thus, a change in the conformation state of β₁ may contribute to LIA. Therefore, we measured total β1 and the β1 activation epitope surface expression by flow cytometry in PBMC±Cdk inihibitors or Jurkat cells±Cdk4 DN. As expected, we found no difference in total surface expression of either β1 or α4 (FIG. 7). In addition, we found no change in expression of β1 activation epitope.

Rb phosphorylation is not required for LIA. The ability of inhibitors to rapidly (within 20 minutes) downregulate LIA suggests that Cdk kinase activity is required for sustained ligand-induced adhesion and transcription is not required. Cdk4 has strict substrate specificity: the only known substrates for Cdk4 are members of the Rb family (Harbour and Dean, Genes Dev 14: 2393-409, 2000) and Smads (primarily Smad 3 and 2). Matsuura et al., Nature 430: 226-31, 2004. To determine whether Rb is involved, we transfected Jurkat cells with a phosphorylation-deficient construct of pRb (pRb^{cdk}) lacking ten Cdk consensus sites. Lukas et al., Genes Dev 11: 1479-92, 1997. Because Rb is unable to be phosphorylated, it remains constitutively active (continues to bind transcription factors and prevents progression through the cell cycle G1. Lukas et al., Oncogene 18: 3930-5, 1999. If Rb phosphorylation is required for LIA, overexpression of this construct should inhibit LIA. However, overexpression of this construct did not inhibit LIA (FIG. 7), suggesting that Rb is not the relevant substrate for Cdk4 mediated adhesion.

Transendothelial migration is inhibited by Cdk inhibitors. To test potential biological relevance of the Cdk4-mediated pathway, we examined the effect of Cdk inhibitors on PBMC migration through a confluent endothelial monolayer. We found a significant decrease of migration in the presence of inhibitors, while initial adhesion was not affected. In addition, endothelial permeability, as indicated by resistance measurements, was similar in all conditions.

### Discussion

We report a new pathway that regulates lymphocyte adhesion to exposed endothelial matrix in the absence of exogenous cytokine or chemokine stimulation. Furthermore, we provide evidence for a novel role of Cdk4 in regulating this adhesion. This provides an unrecognized connection linking cell cycle and adhesion.

We previously reported that high-density FN and staurosporine-treated EC supported spontaneous adhesion of lymphocytes cell lines. Liu et al., J Biol Chem 277: 40893-900, 2002; Schwartz et al., J Immunol 162: 4842-8, 1999. Other examples of regulation of adhesion by ligand have been noted. For example, initial adhesion strength increases with increasing ligand density. Garcia et al., J Biol Chem 273: 10988-93, 1998a; Garcia et al., J Biol Chem 273: 34710-5, 1998b. Eosinophil spreading and migration is inhibited by high density, but not low-density fibronectin. Holub et al., J Leukoc Biol 73: 657-64, 2003. Changes induced by ligand binding can be detected by antibodies that recognize conformational changes on integrins or ligand-induced binding sites (LIBS). Newham et al., J Immunol 160: 4508-17, 1998.

Lymphocyte adhesion to staurosporine-treated EC was mainly α4β1 integrin-dependent. Schwartz et al., J Immunol 162: 4842-8, 1999. Since staurosporine-induced endothelial cell retraction is an early stage of initiating apoptosis, it now appears likely that the induced-adhesion might have been due to exposure of matrix rather than to endothelial cell apoptosis per se. We have several lines of evidence to support this: treatment of EC with agents that induced retraction but did not induce apoptosis increased adhesion of unstimulated cells, and agents that induced apoptosis without causing endothelial cell retraction did not enhance adhesion. Circulating lymphocytes constitutively express α4β1 in a “low-affinity” conformation. We now show that these cells are adhesion-competent when presented with an appropriate matrix. Post-trafficking interactions of lymphocytes with subendoethelial matrix result in “outside-in” signaling that contribute to the inflammatory response. For example, ligand binding of α4β1 can enhance production of several inflammatory mediators such as TNF-α, IL-1 and tissue factor. Kanda et al., Biochemical and Biophysical Research Communications 301: 934-940, 2003; Lin et al., J Biol Chem 270: 16189-97, 1995; McGilvray et al., J Biol Chem 272: 10287-94, 1997. α4β1 binding to VCAM increased β2-mediated cell adhesion. Chan et al., J Immunol 164: 746-53 2000. α1β1 mediated outside-in signaling on lymphocytes regulates cell proliferation, adhesion, migration and activation, which contribute to the inflammatory response. Blockade of α1β1 in animal models of immune-mediated disorders including graft vs. host disease, arthritis, colitis and glomerulonephritis markedly decreased the inflammatory response. In addition, α1−/− mice demonstrate a decreased inflammatory response in several models of immune-mediated inflammation. Reviewed in Ben-Horin and Bank, Clin Immunol 113: 119-29, 2004. Thus, post-trafficking lymphocyte signaling is an important contributor to the inflammatory response. Adhesion of unstimulated lymphocytes to subendothelial matrix suggests an alternate pathway to initiate outside-in mediated inflammatory response.

Many of the features of LIA do not fit into classic adhesion paradigms, including lack of dependence on Rap1, lack of inhibition by cytochalasin, and the role of Cdk in adhesion (see Table 1 below).

TABLE 1
Comparison of two pathways of lymphocyte adhesion

| Stimulated adhesion^{a} | Ligand induced adhesion |
|---|---|
| Rap1-dependent | Rap1-independent |
| Inhibited by cytochalasin D | Not affected by cytochalasin D |
| Beta 1 or beta 2 integrin dependent | Beta 1 integrin dependent |
| Cdk4-independent | Cdk4-dependent |

^{a}i.e. by soluble agonists

The role of Cdk in LIA is supported by three independent lines of evidence: inhibition of LIA by pharmacological inhibitors of Cdk, inhibition of LIA by dominant negative construct of Cdk4, but not Cdk2 and inhibition of LIA by Cdk4 siRNA. In addition, Rap1 dominant negative construct and inhibitors of other pathways failed to block LIA. Cdks are Ser/Thr kinases that regulate progression through the cell cycle. However, there is increasing evidence that Cdks, as well as cyclins and Cdk inhibitors (CKIs) are important for other functions, including cytoskeleton rearrangement and cell migration. Besson et al., Nat Rev Cancer 4: 948-55, 2004. Cdk5 plays an important role in neuronal cell function and can phosphorylate focal adhesion kinase (FAK) and contribute to neurite outgrowth and migration. Gao et al., Mol Cancer Res 1: 12-24, 2002; Negash et al., J Cell Sci 115: 2109-17, 2002. Other Cdks including Cdk 1, 4, 6 regulate neuronal cell death (references). A recent report described a novel role for Cdk1 (cdc2) as a downstream effector of the integrin αvβ3, leading to cell migration. Manes et al., J Cell Biol 161: 817-26, 2003. Furthermore, Cdk1 localized to membrane ruffles of migrating cells. Manes et al., J Cell Biol 161: 817-26, 2003. Thus, there is increasing evidence that Cdks may have non-traditional roles in various cell behaviors, including those related to adhesion and migration.

The major substrate for Cdk4 is Rb. However, our data suggest that known sites of Rb phosphorylation are not required for LIA. Thus, it is likely that eithera novel substrate or

novel Rb phosphorylation site(s) may be involved in LIA. We previously reported that protein synthesis was not required for staurosporine-induced adhesion (Bombeli et al., Blood 93: 3831-8, 1999) and now extended these observations to LIA. It is unknown whether these substrates are involved in LIA, or whether new pathways are involved. Because adhesion is spontaneous, it is unlikely to involve differences in surface expression of β1 integrin. However, change in the conformation state of β1 may occur as a result of spontaneous adhesion.

It is well accepted that cell adhesion can regulate cell cycle progression. In most anchorage-dependent cells, integrin-mediated adhesion is necessary for progression through the cell cycle, with loss of adhesion resulting in G1 phase cell cycle arrest. Furthermore, interactions between integrins and growth factor receptors further potentiate the effect on cell cycle progression. Schwartz and Assoian, J Cell Sci 114: 2553-60, 2001. While it is clear that integrin adhesion regulates cell cycle, little is known about whether the cell cycle regulates integrin adhesion. Our data suggest a potential link in this direction as well.

Finally, we show that LIA contributes to leukocyte trafficking by regulating lymphocyte migration. Since no exogenous stimulation is required for Cdk4 mediated adhesion and migration, this pathway may be involved in constitutive leukocyte trafficking, rather than trafficking in response to inflammatory stimulus.

We show that unstimulated leukocytes are capable of ligand-induced adhesion to high-density ligand or EC matrix. Further characterization of this novel pathway may provide insight into the accompanying posttrafficking events. Ligand-induced adhesion may contribute to leukocyte trafficking and resultant imflammatory response in disorders characterized by abnormal leukocyte infiltration, such as psoriasis, lymphocytic interstitial pneumonitis and graft vs. host disease.

Abbreviations. BAEC: Bovine aortic endothelial cells; DN: Dominant negative; EC: Endothelial cells; FN: Fibronectin; HUVEC: Human umbilical endothelial cells; LIA: ligand induced adhesion; PBMC: Peripheral blood mononuclear cells

When ranges are used herein for physical properties, such as molecular weight, or chemical properties, such as chemical formulae, all combinations and subcombinations of ranges and specific embodiments therein are intended to be included.

The disclosures of each patent, patent application and publication cited or described in this document are hereby incorporated herein by reference, in their entirety.

Those skilled in the art will appreciate that numerous changes and modifications can be made to the embodiments of the invention and that such changes and modifications can be made without departing from the spirit of the invention. It is, therefore, intended that the appended claims cover all such equivalent variations as fall within the true spirit and scope of the invention.

What is claimed:

1. A method for treating an inflammatory-related disease or disorder in a mammalian subject comprising administering to the subject one or more modulators of cyclin dependent kinase 4 (Cdk4) activity in an amount effective to reduce or eliminate the inflammatory-related disease or disorder or prevent its occurrence or recurrence.

2. The method of claim 1 wherein the Cdk4 activity regulates ligand-induced adhesion (LIA).

3. The method of claim 3 wherein the LIA is integrin-mediated.

4. The method of claim 4 wherein the LIA is small GTPase Rap-1 independent.

5. The method of claim 1, wherein the Cdk4 modulator inhibits integrin-mediated adhesion in leukocytes.

6. The method of claim 1, wherein the Cdk4 modulator inhibits integrin-mediated adhesion in monocytes.

7. The method of claim 1 wherein the modulator is a small chemical compound, short interfering RNA, dominant-negative molecule, short hairpin RNA, ribozyme, antisense oligonucleotide, antibody, peptide or peptidomimetic.

8. The method of claim 5 wherein the dominant-negative molecule is a dominant-negative peptide or peptidomimetic.

9. The method of claim 5 wherein the small chemical compound is flavopiridol, indolinone, oxindole 91, N⁶-isopentenyladenine, olomoucine, (R)-Roscovitine, hymenialdisine, fascaplysin, compound 66, purvalanol A,B, indirubin-3′monoxime, indirubin-5-sulfonate, SU9516, compound 26a, compound 15b, alsterpaullone, quinazoline 51, CINK4, PD0183812, NSC 625987, PD 0332991, aminopurvalanol, and Calyculin phosphatase inhibitor.

10. The method of claim 1 wherein the subject is human.

11. The method of claim 1, wherein the inflammatory-related disease or disorder is diabetes, nephropathy, obesity, hearing loss, fibrosis related disease, arthritis, allergy, allergic rhinitis, acute respiratory distress syndrome, asthma, bronchitis, inflammatory bowel disease, an autoimmune disease, hepatitis, atopic dermatitis, pemphigus, glomerulonephritis, atherosclerosis, sarcoidosis, ankylosing spondylitis, Wegner's syndrome, Goodpasture's syndrome, giant cell arteritis, polyarteritis nodosa, idiopathic pulmonary fibrosis, acute lung injury, chronic obstructive pulmonary disease, post-influenza pneumonia, SARS, tuberculosis, malaria, sepsis, cerebral malaria, Chagas disease, schistosomiasis, bacterial and viral meningitis, cystic fibrosis, multiple sclerosis, Alzheimer's disease, encephalomyelitis, sickle cell anemia, pancreatitis, transplantation, systemic lupus erythematosis, thyroiditis, and radiation pneumonitis, lymphocytosis syndrome, or lymphocytic interstitial pneumonitis.

12. The method of claim 11, wherein the diabetes is Type II diabetes, Type I diabetes, diabetes insipidus, diabetes mellitus, maturity-onset diabetes, juvenile diabetes, insulin-dependant diabetes, non-insulin dependant diabetes, malnutrition-related diabetes, autoimmune diabetes, ketosis-prone diabetes or ketosis-resistant diabetes.

13. The method of claim 11, wherein the nephrophaty is glomerulonephritis, acute kidney failure or chronic kidney failure.

14. The method of claim 11, wherein the obesity is hereditary obesity, dietary obesity, hormone related obesity or obesity related to the administration of medication.

15. The method of claim 11, wherein the hearing loss results from otitis extema or acute otitis media.

16. The method of claim 11, wherein the fibrosis related disease is pulmonary interstitial fibrosis, renal fibrosis, cystic fibrosis, liver fibrosis, wound-healing or burn-healing.

17. The method of claim 11, wherein the arthritis is rheumatoid arthritis, rheumatoid spondylitis, psoriatic arthritis, osteoarthritis or gout.

18. The method of claim 11, wherein the irritable bowel disease is irritable bowel syndrome, mucous colitis, ulcerative colitis, Crohn's disease, gastritis, esophagitis, pancreatitis or peritonitis.

19. The method of claim 11 wherein the autoimmune disease is scleroderma, systemic lupus erythematosus, myasthenia gravis, transplant rejection, endotoxin shock, sepsis, psoriasis, eczema, dermatitis or multiple sclerosis.

20. The method of claim 11 wherein the hepatitis is viral chronic hepatitis.

21. A method for reducing or eliminating ligand-induced adhesion in a mammalian subject comprising administering to the subject one or more modulators of cyclin dependent kinase 4 (Cdk4) activity in an amount effective to reduce or eliminate leukocyte adhesion and migration.

22. The method of claim 21 wherein the modulator is a small chemical compound, short interfering RNA, dominant-negative molecule, short hairpin RNA, ribozyme, antisense oligonucleotide, antibody, peptide or peptidomimetic.

23. The method of claim 22 wherein the dominant-negative molecule is a dominant-negative peptide or peptidomimetic.

24. The method of claim 22 wherein the small chemical compound is flavopiridol, indolinone, oxindole 91, N⁶-isopentenyladenine, olomoucine, (R)-Roscovitine, hymenialdisine, fascaplysin, compound 66, purvalanol A, purvalanol B, indirubin-3′monoxime, indirubin-5-sulfonate, SU9516, compound 26a, compound 15b, alsterpaullone, quinazoline 51, CINK4, PD0183812, NSC 625987, PD 0332991, aminopurvalanol, and Calyculin phosphatase inhibitor.

25. The method of claim 21 wherein the subject is human.

26. A composition comprising a therapeutically effective amount of at least one modulator of cyclin dependent kinase 4 (Cdk4) activity for treatment of an inflammatory-related disease or disorder in a mammalian subject.

27. The composition of claim 26 wherein the therapeutically effective amount is a prophylactically effective amount.

28. The composition of claim 26 wherein the Cdk4 activity regulates ligand-induced adhesion (LIA).

29. The composition of claim 28 wherein the LIA is integrin-mediated.

30. The composition of claim 29 wherein the LIA is small GTPase Rap-1 independent.

31. The composition of claim 26 wherein the Cdk4 modulator inhibits integrin-mediated adhesion in leukocytes.

32. The composition of claim 26 wherein the Cdk4 modulator inhibits integrin-mediated adhesion in monocytes.

33. A pharmaceutical composition comprising at least one Cdk4 modulator of claim 26 and a pharmaceutically acceptable carrier.

34. The composition of claim 33 wherein the inflammatory-related disease or disorder wherein the inflammatory-related disease or disorder is diabetes, nephropathy, obesity, hearing loss, fibrosis related disease, arthritis, allergy, allergic rhinitis, acute respiratory distress syndrome, asthma, bronchitis, inflammatory bowel disease, an autoimmune disease, hepatitis, atopic dermatitis, pemphigus, glomerulonephritis, atherosclerosis, sarcoidosis, ankylosing spondylitis, Wegner's syndrome, Goodpasture's syndrome, giant cell arteritis, polyarteritis nodosa, idiopathic pulmonary fibrosis, acute lung injury, chronic obstructive pulmonary disease, post-influenza pneumonia, SARS, tuberculosis, malaria, sepsis, cerebral malaria, Chagas disease, schistosomiasis, bacterial and viral meningitis, cystic fibrosis, multiple sclerosis, Alzheimer's disease, encephalomyelitis, sickle cell anemia, pancreatitis, transplantation, systemic lupus erythematosis, thyroiditis, and radiation pneumonitis, lymphocytosis syndrome, or lymphocytic interstitial pneumonitis.

35. The composition of claim 34 wherein the diabetes is Type II diabetes, Type I diabetes, diabetes insipidus, diabetes mellitus, maturity-onset diabetes, juvenile diabetes, insulin-dependant diabetes, non-insulin dependant diabetes, malnutrition-related diabetes, autoimmune diabetes, ketosis-prone diabetes or ketosis-resistant diabetes.

36. The composition of claim 34 wherein the nephrophaty is glomerulonephritis, acute kidney failure or chronic kidney failure.

37. The composition of claim 34 wherein the obesity is hereditary obesity, dietary obesity, hormone related obesity or obesity related to the administration of medication.

38. The composition of claim 34 wherein the hearing loss results from otitis extema or acute otitis media.

39. The composition of claim 34 wherein the fibrosis related disease is pulmonary interstitial fibrosis, renal fibrosis, cystic fibrosis, liver fibrosis, wound-healing or burn-healing.

40. The composition of claim 34 wherein the arthritis is rheumatoid arthritis, rheumatoid spondylitis, psoriatic arthritis, osteoarthritis or gout.

41. The composition of claim 34 wherein the irritable bowel disease is irritable bowel syndrome, mucous colitis, ulcerative colitis, Crohn's disease, gastritis, esophagitis, pancreatitis or peritonitis.

42. The composition of claim -34 wherein the autoimmune disease is scleroderma, systemic lupus erythematosus, myasthenia gravis, transplant rejection, endotoxin shock, sepsis, psoriasis, eczema, dermatitis or multiple sclerosis.

43. The composition of claim 34 wherein the hepatitis is viral chronic hepatitis.

44. The composition of claim 33 wherein the modulator is interfering RNA, short hairpin RNA, ribozyme, antisense oligonucleotide, or protein inhibitor.

45. The composition of claim 33 wherein the modulator is a dominant-negative molecule, peptide, peptidomimetic or a small chemical molecule.

46. The composition of claim 45 wherein the dominant-negative molecule is a dominant-negative peptide or peptidomimetic.

47. The composition of claim 45 wherein the small chemical compound is flavopiridol, indolinone, oxindole 91, N⁶-isopentenyladenine, olomoucine, (R)-Roscovitine, hymenialdisine, fascaplysin, compound 66, purvalanol A, purvalanol B, indirubin-3′monoxime, indirubin-5-sulfonate, SU9516, compound 26a, compound 15b, alsterpaullone, quinazoline 51, CINK4, PD0183812, NSC 625987, PD 0332991, aminopurvalanol, and Calyculin phosphatase inhibitor.

48. The composition of claim 33, wherein the composition is administered orally, topically or systemically.
